(19) United States
(12) Patent Application Publication
(10) Pub. No.: US 2025/0204828 A1
(43) Pub. Date: Jun. 26, 2025

(54) SYSTEM AND COMPUTER-IMPLEMENTED METHOD FOR MACHINE LEARNING MODELLING OF USER INTERFACE INTERACTION

(71) Applicant: STELLENBOSCH UNIVERSITY, Stellenbosch (ZA)

(72) Inventors: Pieter Rousseau Fourie, Durbanville (ZA); Romano Swarts, Cape Town (ZA)

(21) Appl. No.: 19/074,931

(22) Filed: Mar. 10, 2025

Related U.S. Application Data
(63) Continuation-in-part of application No. 16/771,371, filed on Jun. 10, 2020.

(30) Foreign Application Priority Data
Dec. 11, 2017 (ZA) 2017/08360
Apr. 3, 2018 (ZA) 2018/02104

(51) Int. Cl. A61B 5/16; A61B 5/00; G16H 50/20; G16H 50/50

(52) U.S. Cl. CPC A61B 5/168; A61B 5/7475; G16H 50/20; G16H 50/50; A61B 2503/06; A61B 2560/0487

(57) ABSTRACT

A system and computer-implemented method for machine learning modelling of user interface interaction are provided. The method includes training a collection of machine learning algorithms using training data to discriminate between users with and without a neurological condition by identifying patterns in a feature set which are indicative of the presence or absence of the condition and labelling the feature set accordingly. The collection of machine learning algorithms includes a locally deep support vector machine-based algorithm and a consensus algorithm. The training includes: receiving, from a mobile communication device, a payload including recorded data points; compiling at least a subset of the recorded data points into a feature set; and learning from the compiled feature set by reference to a provided pre-labelled feature set labelled with a condition of a user who caused generation of data points from which the pre-labelled feature set is compiled.

### CROSS-REFERENCE(S) TO RELATED APPLICATIONS

This application is a continuation-in-part of U.S. patent application Ser. No. 16/771,371, filed on 10 Jun. 2020, which is a U.S. National Phase Application pursuant to 35 U.S.C. § 371 of International Application number PCT/IB2018/05986, which claims priority to South African provisional patent application number 2017/08360, filed on 11 Dec. 2017, and South African provisional patent application number 2018/02104, filed on 3 Apr. 2018, the entire disclosure contents of these applications are herewith incorporated by reference for all purposes into the present application.

### TECHNICAL FIELD

This disclosure relates generally to the field of machine learning, and particularly to pattern recognition and classification. More particularly, this disclosure relates to data collection for, and training of, machine learning algorithms including classification algorithms relating to user interface interaction. Even more particularly, this disclosure relates to software arrangements specially adapted for data collection for machine learning algorithms including classification algorithms. Data may be collected and the machine learning algorithms may be trained for discriminating between users with and without a neurological condition.

### BACKGROUND

Attention deficit disorder (with or without hyper-activity) typically presents with problems such as lack of concentration, inattentiveness, poor memory, no sense of time, poor social skills and low self-esteem. The incidence of ADD/ADHD and autism spectrum disorders varies between populations, as well as within social groups, but typically can be expected to be between 2% and 5% of populations. The aetiology of ADD/ADHD has been linked to a decrease of dopamine in the prefrontal cortex.

The management of ADD/ADHD includes diet management (e.g. supplementation with 3 omega, 6 omega fatty acids, coupled with low sugar in-take), occupational therapy, biofeedback, brain gym and the like. Drugs such as methylphenidate (stimulant) and atomoxifene (non-stimulant) have been demonstrated to be effective. However, side effects such as personality changes, headaches, abnormal discomfort, tick disorders as well as high cost limit the use of these medications. Also, because of a wide variability in response to the medication, the exact dosage is often determined by trial and error.

The screening of ADD/ADHD is to a large extent based on subjective techniques such as the Conner's or Swan reports, psychological assessment, and feedback by parents and teachers. To date, no objective (quantitative) technique exists whereby the screening or drug effectiveness can be demonstrated with any degree of accuracy.

It is most desirable to ascertain the effectiveness of medication for any one or more or a variety of different reasons including in order to determine the correct identification of ADD or ADHD; to limit side effects due to overdose; to curb costs; to determine whether the dose should be increased as a child grows older; and to determine which drug works more effectively, for example, stimulant versus non-stimulant types of drugs.

The above challenges may be compounded by the lack of access to medical professionals and medical technology that is typically experienced in rural and/or developing regions across the globe. The challenge will be the identification and monitoring of children with attention deficit hyperactivity disorder, autism or other neuro-developmental disorders.

The preceding discussion of the background is intended only to facilitate an understanding of the present disclosure. It should be appreciated that the discussion is not an acknowledgment or admission that any of the material referred to was part of the common general knowledge in the art as at the priority date of the application.

### SUMMARY

In accordance with an aspect of the disclosure there is provided a computer-implemented method for machine learning modelling of user interface interaction comprising: training a collection of machine learning algorithms using training data to discriminate between users with and without a neurological condition by identifying patterns in a feature set which are indicative of the presence or absence of the condition and labelling the feature set accordingly, wherein the collection of machine learning algorithms includes a locally deep support vector machine-based algorithm and a consensus algorithm; the training including: receiving, from a mobile communication device on which a virtual environment and a plurality of environment-based discriminator events are output via a multi-touch-sensitive display thereof, a payload including recorded data points having been generated by recording and time stamping each user interface input instruction input into the mobile communication device via the multi-touch-sensitive display by a user of the mobile communication device and by recording and time stamping the output of the environment-based discriminator events such that the recorded data points are mapped to the discriminator events and relate to the user's interaction with the mobile communication device in relation to each of the plurality of environment-based discriminator events; compiling at least a subset of the recorded data points into a feature set, wherein the feature set is designed to define features used to classify the neurological condition, wherein the feature set is partitioned to delineate features obtained from each of one or more segments of the virtual environment, wherein each of the one or more segments includes one or more of the plurality of environment-based discriminator events designed to test an aspect of the neurological condition; and, learning from the compiled feature set for a segment of the one or more segments by reference to a provided pre-labelled feature set labelled with a condition of a user who caused generation of data points from which the pre-labelled feature set is compiled.

The method may include labelling the feature set with the condition of the user who caused generation of data points from which the feature set is compiled to generate the pre-labelled feature set. The pre-labelled feature set may be associated with one or more segments of the virtual environment. The pre-labelled feature set may be labelled with one or more identifiers of the segments from which it was obtained.

The method may include continually training the collection of machine learning algorithms with classification feedback.

Compiling at least a subset of the recorded data points into the feature set may include the subset of recorded data points representing first order features. The method may include: processing the first order features to generate second order features; and, including at least a subset of the second order features together with the subset of the first order features in the feature set.

The locally deep support vector machine-based algorithm may be configured to classify the feature set based on patterns included therein. The locally deep support vector machine-based algorithm may process the features obtained from each of the one or more segments to obtain the classification which corresponds to the segment of the one or more segments.

Training may include training the locally deep support vector machine-based algorithm using data points obtained from each of the one or more segments.

The method may include a diagnostic method of: receiving, from a mobile communication device on which the virtual environment and environment-based discriminator events are output via a display thereof, a payload including recorded data points and a user identifier uniquely identifying a user-under-test, the recorded data points having been generated by recording and time stamping each user interface input instruction received from the user-under-test and by recording and time stamping the output of the environment-based discriminator events such that the recorded data points are mapped to the discriminator events and relate to the user's interaction with the mobile communication device in relation to each of the plurality of environment-based discriminator events; compiling at least a subset of the recorded data points into a feature set, wherein the feature set is designed to define features used to classify the neurological condition, wherein the feature set is partitioned to delineate features obtained from each of the one or more segments; processing the feature set by the locally deep support vector machine-based algorithm, including: processing features obtained from each of the one or more segments by the locally deep support vector machine-based algorithm to obtain a classification for that segment; processing each of the classifications by the consensus algorithm to determine a label indicating either the presence or absence of the condition; and, outputting the label in association with the user identifier, including transmitting, via a communication network, the label to one or both of the mobile communication device from which the data points are received and to a communication device of a medical practitioner linked to the user identifier.

The method may include associating one or more of the recorded data points, the feature set and the label with a user record linked to the user identifier; and, monitoring changes in the recorded data points and labels associated with the user record. The condition may be linked to a spectrum and the label may indicate either the presence or absence of the condition by indicating a region of the spectrum with which the feature set is associated.

The method may include compiling the payload including the recorded data points and the user identifier with the recorded data points mapped to the discriminator events; and, transmitting the payload to a server computer or processing the payload on the mobile communication device for compilation into the feature set for the collection of machine learning algorithms, wherein the feature set represents measured features used by the collection of machine learning algorithms to classify users with and without the neurological condition by identifying patterns in the feature sets.

In accordance with a further aspect of the disclosure there is provided a computer-implemented method for machine learning modelling of user interface interaction comprising: outputting a virtual environment on a mobile communication device to a user, the virtual environment being output to the user via one or more output components of the mobile communication device including a multi-touch-sensitive display with which the user interacts by inputting a series of instructions into at least one input interface including the multi-touch-sensitive display of the mobile communication device; outputting a plurality of environment-based discriminator events in the virtual environment; recording data points relating to the user's interaction with the mobile communication device in relation to each of the discriminator events including recording and time stamping each user interface input instruction input into the mobile communication device via the multi-touch-sensitive display by the user and recording and time stamping the output of the environment-based discriminator events such that the recorded data points are mapped to the discriminator events and relate to the user's interaction with the mobile communication device in relation to each of the plurality of environment-based discriminator events; sending to a collection of trained machine learning algorithms for discriminating between users with and without a neurological condition, from the mobile communication device, a payload including the recorded data points.

The virtual environment may include one or more segments. Different segments may include different environment-based discriminator events for testing an aspect of the neurological condition. The virtual environment may include a virtual character. Each of the one or more segments may include a plurality of discriminator events for testing an aspect of the neurological condition. The user interaction may include controlling navigation of the virtual character through a segment of the one or more segments. One or more segments of the one or more segments may be in the form of a mini-game and may include a number of difficulty levels associated therewith. Each of the environment-based discriminator events may include one or more of: a stimulus output element provided in the virtual environment and output from the mobile communication device to the user, wherein the stimulus output element is configured to prompt a predetermined expected instruction input into the mobile communication device by the user; a distractor output element provided in the virtual environment and output from the mobile communication device to the user, the distractor output element being configured to distract the user from required interaction with the virtual environment; and, a pause or exit input element configured upon activation to pause or exit the virtual environment. Recording data points relating to the user's interaction in relation to the environment-based discriminator event in the form of the stimulus output element includes one or more of: recording a time stamp corresponding to a time at which the stimulus output element is output from the mobile communication device to the user; recording a time stamp corresponding to a time at which the user inputs an input instruction in response to the output of the stimulus output element; and, evaluating the input instruction received in response to the output of the stimulus output element against the predetermined expected instruction input.

The method may be conducted by a mobile software application downloadable onto and executable on the mobile communication device. The mobile software application may be downloadable from an application repository maintained by a third party. The mobile software application may provide the virtual environment in the form of a computer game. Recording data points may include recording motion data produced by motion sensors associated with the mobile communication device for recording the movement of the mobile communication device. The motion data may relate to acceleration and/or rotation data produced by an accelerometer or gyroscope respectively, wherein the recorded motion data is position-stamped and/or time-stamped.

In accordance with a further aspect of the disclosure there is provided a system for machine learning modelling of user interface interaction comprising a non-transitory computer-readable storage medium, and one or more processors coupled to the non-transitory computer-readable storage medium. The non-transitory computer-readable storage medium comprises program instructions that, when executed on the one or more processors, cause the system to perform operations comprising: training a collection of machine learning algorithms using training data to discriminate between users with and without a neurological condition by identifying patterns in a feature set which are indicative of the presence or absence of the condition and labelling the feature set accordingly, wherein the collection of machine learning algorithms includes a locally deep support vector machine-based algorithm and a consensus algorithm; the training including: receiving, from a mobile communication device on which a virtual environment and a plurality of environment-based discriminator events are output via a multi-touch-sensitive display thereof, a payload including recorded data points having been generated by recording and time stamping each user interface input instruction input into the mobile communication device via the multi-touch-sensitive display by a user of the mobile communication device and by recording and time stamping the output of the environment-based discriminator events such that the recorded data points are mapped to the discriminator events and relate to the user's interaction with the mobile communication device in relation to each of the plurality of environment-based discriminator events; compiling at least a subset of the recorded data points into a feature set, wherein the feature set is designed to define features used to classify the neurological condition, wherein the feature set is partitioned to delineate features obtained from each of one or more segments of the virtual environment, wherein each of the one or more segments includes one or more of the plurality of environment-based discriminator events designed to test an aspect of the neurological condition; and, learning from the compiled feature set for a segment of the one or more segments by reference to a provided pre-labelled feature set labelled with a condition of a user who caused generation of data points from which the pre-labelled feature set is compiled.

In accordance with a further aspect of the disclosure there is provided a computer-implemented method comprising: providing a virtual environment which is output to a user via one or more output components of a communication device and with which the user is required to interact by way of a series of instructions input into the communication device, wherein the virtual environment includes a number of environment-based discriminators which based on a user's interaction relative thereto facilitate discrimination between a user with and without a condition; recording data points relating to the user's interaction in relation to each of the number of environment-based discriminators; compiling a payload including the recorded data points and a user identifier; and outputting the payload for input into a machine learning component configured to discriminate between users with and without the condition by identifying patterns in the data points.

A further feature provides for recording parameters relating to the user's interaction includes using one or more of a clock, timer and trigger within the virtual environment. Further features provide for the method to be conducted by a mobile software application downloadable onto and executable on the communication device; and for the mobile software application to be downloadable from an application repository maintained by a third party.

Still further features provide for the virtual environment to include a virtual character and a segment; for the user interaction to include controlling navigation of the virtual character through the segment; for the virtual environment to include a plurality of segments; for each segment to include a number of environment-based discriminators; for different segments to include different environment-based discriminators for facilitating discrimination between users with and without different conditions; for the virtual environment to provide an open world environment in which the user can navigate the virtual character between different segments; and, for the virtual environment to include adaptive segments. One or more segments may be in the form of a mini-game and may include a number of difficulty levels (e.g. degrees of difficulty) associated therewith. Each segment may be configured to facilitate discrimination between a different condition that may be associated with neuro-developmental disorders (attention deficit disorder (ADD), attention deficit hyperactivity disorder (ADHD), ADHD inattention subtype, ADHD hyperactive/impulsive subtype, autism spectrum disorders (ASD), etc.).

A yet further feature provides for each of the number of environment-based discriminators to include one or more of: a stimulus output element provided in the virtual environment and output from the communication device to the user, wherein the stimulus output element may be configured to prompt a predetermined expected instruction input into the communication device by the user; a distractor output element provided in the virtual environment and output from the communication device to the user, the distractor output element being configured to distract the user from required interaction with the virtual environment; a pause or exit input element configured upon activation to pause or exit the virtual environment.

A further feature provides for recording data points relating to the user's interaction in relation to an environment-based discriminator in the form of a stimulus output element to include one or more of: recording a time stamp corresponding to the time at which the stimulus output element is output from the communication device to the user; recording a time stamp corresponding to the time at which the user inputs an input instruction in response to output of the stimulus output element; and, evaluating an input instruction received in response to output of the stimulus output element against the predetermined expected instruction input.

Still further features provide for one of the number of environment-based discriminators to include a number of stimulus output elements, and for recording parameters relating to the user's interaction relative to an environment-based discriminator in the form of a number of stimulus output elements to include tracking a trajectory of the virtual character through the virtual environment in relation to the location of the stimulus output element in the virtual environment. An even further feature provides for compiling the payload to include associating the recorded data points with the environment-based discriminator in relation to which they were recorded.

In accordance with a further aspect of the disclosure there is provided a computer-implemented method comprising: receiving, from a communication device, a payload including recorded data points and a user identifier uniquely identifying a user, wherein the data points relate to the user's recorded interaction with a virtual environment in relation to each of a number of environment-based discriminators included within the virtual environment, wherein the environment-based discriminators and the user's interaction relative thereto facilitate discrimination between a user with and without a condition, wherein the virtual environment is output to the user via one or more output components of the communication device and wherein the user is required to interact with the virtual environment by way of a series of instructions input into the communication device; inputting a feature set including at least a subset of the data points into a machine learning component configured to discriminate between users with and without the condition by identifying patterns in the feature set which are indicative of the presence or absence of the condition and labelling the feature set accordingly; receiving a label from the machine learning component indicating either the presence or absence of the condition; and outputting the label in association with the user identifier.

Further features provide for the method to include compiling at least a subset of the data points into a feature set, wherein the subset of data points represent first order features and wherein the method includes: processing the first order features to generate second order features; and, including at least a subset of the second order features together with the subset of the first order features in the feature set.

Yet further features provide for the machine learning component to include a classification component configured to classify the feature set based on patterns included therein; for the machine learning component to include a plurality of classification components and a consensus component, for each of the plurality of classification components to be associated with a corresponding segment of the virtual environment, for the feature set to be partitioned to delineate features obtained from each of the segments, and for inputting the feature set into the machine learning component to include: inputting features obtained from a particular segment into the associated classification component; receiving a classification from each classification component which corresponds to each of the segments; inputting each of the classifications into the consensus component, wherein the consensus component evaluates the classifications of each of the classification components and outputs a label indicating either the presence or absence of the condition based on the consensus; and, receiving a label from the consensus component; for each classification component to be trained using data points obtained from the segment of the virtual environment with which it is associated; and, for the or each classification component to implement a neural network-, boosted decision tree-or locally deep support vector machine-based algorithm.

A further feature provides for the method to include associating one or both of the recorded data points, the feature set and the label with a user record linked to the user identifier. A still further feature provides for the method to include monitoring changes in the recorded data points and labels associated with the user record. A yet further feature provides for the method to include training the machine learning component using training data including a pre-labelled feature set. An even further feature provides for the condition to be linked to a spectrum and for the label to indicate either the presence or absence of the condition by indicating a region of the spectrum with which the feature set is associated.

In accordance with a further aspect of the disclosure there is provided a system including a memory for storing computer-readable program code and a processor for executing the computer-readable program code, the system comprising: a virtual environment providing component for providing a virtual environment which is output to a user via one or more output components of a communication device and with which the user is required to interact by way of a series of instructions input into the communication device, wherein the virtual environment includes a number of environment-based discriminators which based on a user's interaction relative thereto facilitate discrimination between a user with and without a condition; a data point recording component for recording data points relating to the user's interaction in relation to each of the number of environment-based discriminators; a compiling component for compiling a payload including the recorded data points and a user identifier; and an outputting component for outputting the payload for input into a machine learning component configured to discriminate between users with and without the condition by identifying patterns in the data points.

In accordance with a further aspect of the disclosure there is provided a system including a memory for storing computer-readable program code and a processor for executing the computer-readable program code, the system comprising: a receiving component for receiving, from a communication device, a payload including recorded data points and a user identifier uniquely identifying a user, wherein the data points relate to the user's recorded interaction with a virtual environment in relation to each of a number of environment-based discriminators included within the virtual environment, wherein the environment-based discriminators and the user's interaction relative thereto facilitate discrimination between a user with and without a condition, wherein the virtual environment is output to the user via one or more output components of the communication device and wherein the user is required to interact with the virtual environment by way of a series of instructions input into the communication device; a feature set inputting component for inputting a feature set including at least a subset of the data points into a machine learning component configured to discriminate between users with and without the condition by identifying patterns in the feature set which are indicative of the presence or absence of the condition and labelling the feature set accordingly; a label receiving component for receiving a label from the machine learning component indicating either the presence or absence of the condition; and an outputting component for outputting the label in association with the user identifier.

In accordance with a further aspect of the disclosure there is provided a computer program product comprising a computer-readable medium having stored computer-readable program code for performing the steps of: providing a virtual environment which is output to a user via one or more output components of a communication device and with which the user is required to interact by way of a series of instructions input into the communication device, wherein the virtual environment includes a number of environment-based discriminators which based on a user's interaction relative thereto facilitate discrimination between a user with and without a condition; recording data points relating to the user's interaction in relation to each of the number of environment-based discriminators; compiling a payload including the recorded data points and a user identifier; and outputting the payload for input into a machine learning component configured to discriminate between users with and without the condition by identifying patterns in the data points.

In accordance with a further aspect of the disclosure there is provided a computer program product comprising a computer-readable medium having stored computer-readable program code for performing the steps of: receiving, from a communication device, a payload including recorded data points and a user identifier uniquely identifying a user, wherein the data points relate to the user's recorded interaction with a virtual environment in relation to each of a number of environment-based discriminators included within the virtual environment, wherein the environment-based discriminators and the user's interaction relative thereto facilitate discrimination between a user with and without a condition, wherein the virtual environment is output to the user via one or more output components of the communication device and wherein the user is required to interact with the virtual environment by way of a series of instructions input into the communication device; inputting a feature set including at least a subset of the data points into a machine learning component configured to discriminate between users with and without the condition by identifying patterns in the feature set which are indicative of the presence or absence of the condition and labelling the feature set accordingly; receiving a label from the machine learning component indicating either the presence or absence of the condition; and outputting the label in association with the user identifier.

Further features provide for the computer-readable medium to be a non-transitory computer-readable medium and for the computer-readable program code to be executable by a processing circuit.

Embodiments of the disclosure will now be described, by way of example only, with reference to the accompanying drawings.

### BRIEF DESCRIPTION OF THE DRAWINGS

FIG. 1A is a schematic diagram which illustrates data flow from a communication device to a machine learning component according to aspects of the present disclosure.

FIG. 1B is a schematic diagram which illustrates data flow from a communication device to a database according to aspects of the present disclosure.

FIG. 1C is a schematic diagram which illustrates an exemplary system for screening for and monitoring a condition.

FIG. 2A is a schematic diagram which illustrates an exemplary virtual environment including a virtual character according to aspects of the present disclosure.

FIGS. 2B, 2C, and 2D are screenshots of the virtual environment as it may be displayed by a communication device.

FIG. 3 is a swim-lane flow diagram illustrating an exemplary method for screening for and monitoring a condition.

FIG. 4 is a block diagram which illustrates exemplary components which may be provided by a system for screening for and monitoring a condition.

FIG. 5A a schematic representation of a feature set according to aspects of the present disclosure.

FIG. 5B is a continuation of the feature set of FIG. 5A.

FIG. 6 illustrates an exemplary mapping of features to DSM-V criteria according to aspects of the present disclosure.

FIG. 7 illustrates an example of a computing device in which various aspects of the disclosure may be implemented.

### DETAILED DESCRIPTION

Aspects of this disclosure are directed towards screening for and monitoring one or more conditions. The condition may be a medical condition. In some aspects, a mobile software application provides an open world virtual environment through which a user can, by way of instructions, input into a communication device, executing the application, navigate a virtual character between multiple different segments (or “mini-games”). In some implementations, different segments include different discriminators which are configured to facilitate discrimination between users with and without a particular condition, based on how the user interacts with the environment based discriminators. Thus, a multitude of segments may be provided, each of which causing interaction by the user which is indicative of the presence or absence of a condition, as the case may be. The user's interaction relative to the discriminators may be recorded and transmitted to a remote server computer for processing to identify and monitor the one or more conditions.

The term “open world” as used herein may refer to a video game where a user can move freely through a virtual world and is given considerable freedom in regard to how and when to approach particular segments or objectives, and may be contrasted with other video games that have a more linear structure to their gameplay.

The term “segment” as used herein may refer to the total space available to the user in the virtual environment during the course of completing a discrete objective. Synonyms for “segment” may include “mini-game” map, area, stage, track, board, zone, or phase.

In some implementations, monitoring of drug effectiveness may be provided, especially, but not exclusively in the treatment of attention deficit disorders, e.g. ADD and ADHD, as defined according to the Diagnostic and Statistical Manual-(DSM-) 5 classification. DSM-5 (formerly known as DSM-V) is the fifth edition of the American Psychiatric Association's (APA) Diagnostic and Statistical Manual of Mental Disorders. In the USA the DSM serves, as far as applicant is aware, as a universal authority for the screening for or identification of psychiatric disorders. Treatment recommendations, as well as payment by health care providers, are often determined by DSM classifications.

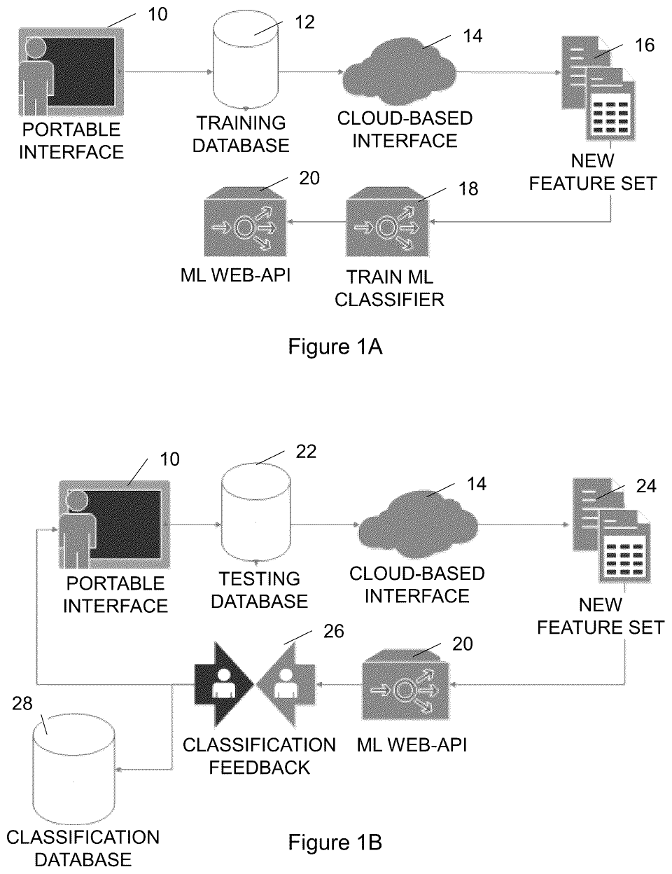

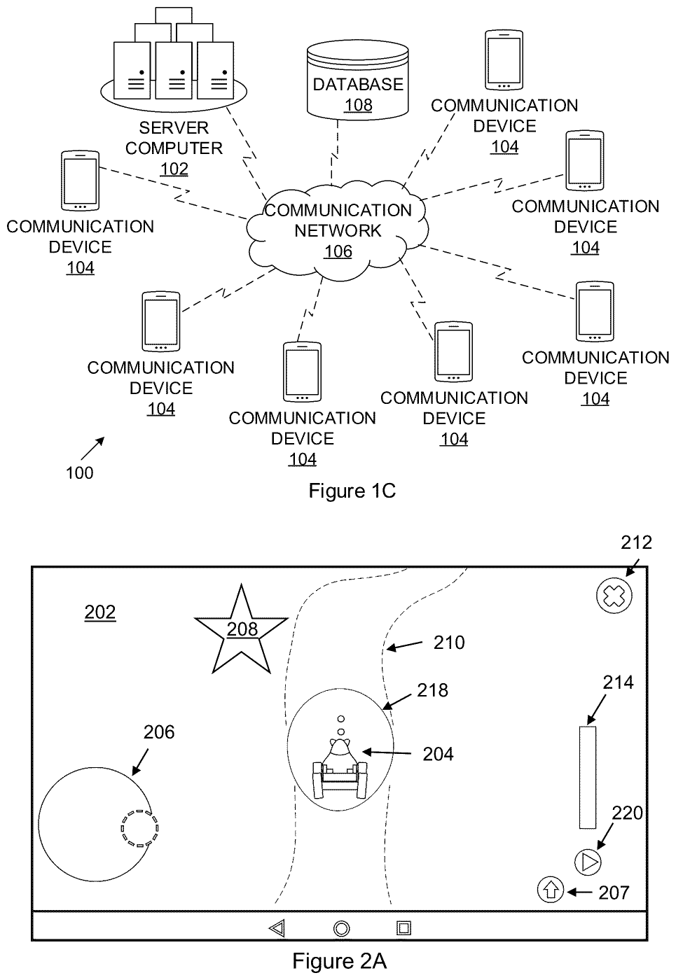

FIG. 1 is a schematic diagram which illustrates an exemplary system (100) for screening for and monitoring a condition.

In FIG. 1A, after a user successfully navigates the virtual environment (i.e. completes the game) on a portable interface (10) provided by a communication device, data points relating to game data and having been recorded during game play are uploaded to a training database (12). This data may then be extracted by a cloud-based interface (14) and processed to create a new feature set (16). The new feature set may be used to train (18) the machine learning component (particularly the classifier) and create a Web-API (20).

In FIG. 1B, after a user exits or completes the game on the portable interface (10), data points relating to game data, or sections of the game data, are automatically uploaded to a testing database (22). This data is then extracted by the cloud-based interface (14) and processed to create a new feature set (24). Creating the feature set may include including in the feature set first order features in the form of data points received from the portable interface as well as second order features, which may be derived from the first order features. In some implementations, in order to derive or create the second-order features for classification or labelling of user data points, all the samples used to train the latest machine learning component (and any machine learning classifiers thereof) may be required to be imported into to the testing database. This may be required to scale the testing participant's vector data according to the vector data samples of the other participants in the training database and in turn to enable principle component analysis (PCA) to be performed on the testing participant's vector data. The cloud-based interface scales and performs PCA with automated scripts. The Web-API (20) is used to classify the new feature set (24). The classification feedback (26) from the Web-API (20) is stored in the classification database (28) and can also be presented on multiple electronic interfaces.

FIG. 1C of the schematic diagram of the system, system (100) may include a server computer (102) and a plurality of communication devices (104). The communication devices (104) may be spread over large geographical regions, potentially across the globe, and may be configured to communicate with the server (102) by way of an appropriate communication network (106), such as a wired or wireless local or wide area network (including, e.g. the Internet).

The communication devices (104) may be computing devices capable of communicating with the server (102) via the communication network (106). Some or all of the communication devices (104) may be portable, handheld communication devices. Each communication device may include a multi-touch-sensitive display, speakers, wireless connectivity and motion sensors (such as a three-axis accelerometer and/or gyroscope). Exemplary communication devices include mobile phones, tablet computers, wearable computing devices, virtual reality headsets, gaming consoles, desktop or laptop computers, smart appliances and the like. Each communication device (104) may include one or more output components via which a virtual environment may be output to a user thereof. Exemplary output components may include a display (e.g. multi-touch sensitive display), speaker, haptic (e.g. vibrator) output component and the like.

Some of the plurality of communication devices (104) may personally belong to respective users while others may be provided for communal use (e.g. in a classroom, etc.). The communication devices may be configured to download and execute a mobile software application providing a virtual environment. The mobile software application may be downloadable from an application repository provided by a third party.

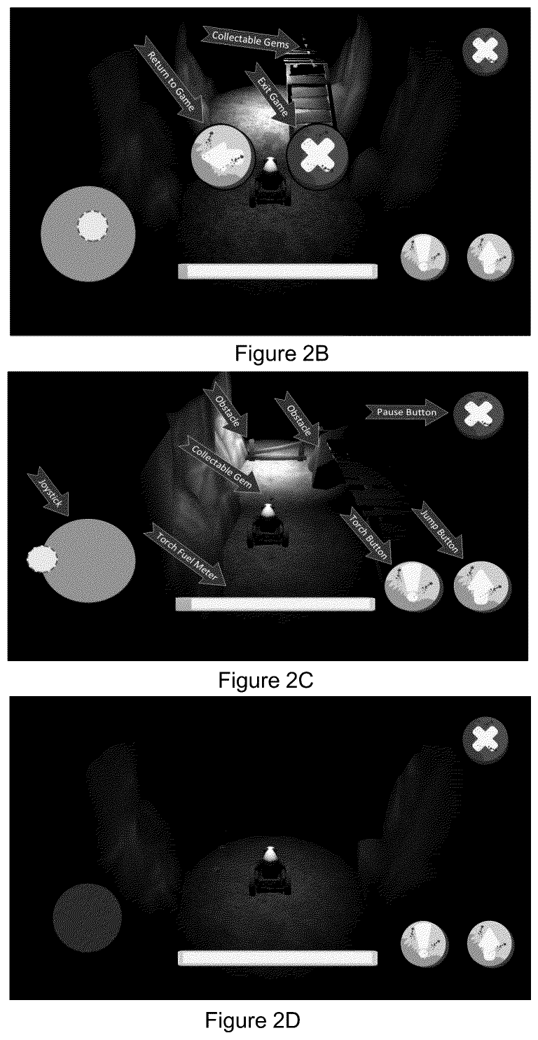

The mobile software application may provide a virtual environment which is in the form of or resembles a video game or computer game. An exemplary virtual environment (202) is illustrated in FIG. 2A to 2D. The virtual environment may include a virtual character (204) and a number of environment-based discriminators which facilitate discrimination between a user with and without a particular condition based on how the user interacts with the discriminator.

The environment-based discriminators may be any element or characteristic of the virtual environment which causes or induces a particular input or response from a user with a particular condition and which input or response would be different for another user who does not have that particular condition. Environment-based discriminators are elaborated on below and may be present in various forms, including, for example, a particular arrangement of game assets (e.g. long, boring tunnel, winding path, obstacles, etc.) collectable gems, obstacles, visual distractors, auditory distractors, so-called ‘kamikaze’ gems and the like. The environment-based discriminators may aid a machine learning component in screening for conditions in its classification and confidence of that classification.

The virtual environment (202) may include an instruction input element (206) configured to receive a user's input instructions for interacting with the virtual environment. In the illustrated example, the instruction input element (206) is in the form of a joystick displayed to the user via a touch sensitive display of the communication device and via which the user can control the virtual character (204). In some implementations there may be other input elements (207) for controlling the virtual character, for example a ‘jump’ input element which is configured to cause the virtual character to jump over an obstacle, etc.

In some implementations, the virtual environment may be custom built for the purpose of screening for and/or identifying a particular condition and the environment-based discriminators may be predetermined discriminators which are purposefully provided within the environment to induce or elicit a particular input or response from the user. In other implementations, the virtual environment may be provided by a general purpose video or computer game which implicitly includes environment-based discriminators provided for screening for and/or identifying a particular condition.

One example of an environment-based discriminator which may be provided in the virtual environment is a stimulus output element. A stimulus output element may be an object configured for output from the communication device to the user (e.g. via display, speaker, etc.). The stimulus output element may be configured to prompt a predetermined expected instruction to be input into the communication device by the user. For example, in one embodiment, where the virtual environment includes a character (204) which the user is required to control or guide along a path, a stimulus output element in the form of a ‘collectable item’ (208) may be provided. The predetermined expected instruction associated with such a stimulus output element may include a series of input instructions which cause traversal of the virtual character (204) from its current position in the virtual environment towards the position of the stimulus output element (or ‘collectable item’). In another scenario the stimulus output element may be a hazard or obstacle which the user is expected to cause the character (through appropriate input instructions) to avoid. In such a case, the predetermined expected instruction associated with the stimulus output element may include a series of instructions which cause traversal of the character away from or clear of the stimulus output element.

Another example of an environment-based discriminator which may be provided in the virtual environment is a distractor output element. A distractor output element may be provided in the virtual environment and output from the communication device to the user. The distractor output element may be configured to distract the user from required interaction with the virtual environment (i.e. to distract the user from what he or she should otherwise be doing). Distractor output elements may be provided in conjunction with, for example, stimulus output elements so as to distract the user from, for example, collecting a collectable item or the like. In the example illustrated in FIG. 2A, the distractor output element may be configured to distract the user from the task of navigating the virtual character (204) along the pathway (210) which the virtual character is required to traverse.

Yet another example of an environment-based discriminator may be a pause or exit input element (212) configured upon activation to pause or exit the virtual environment.

There may be other types of environment-based discriminators. For example, in the exemplary implementations illustrated in FIGS. 2A to 2D, ‘collectable gems’ may have the effect of refuelling or recharging a torch meter (214) which may in turn allow a torch carried by the virtual character (204) to be used to illuminate (218) better the virtual environment (202). The torch may be toggled on and off via an appropriate input element (220). The torch illuminating the virtual environment is illustrated in the screenshots of FIGS. 2B and 2C while FIG. 2D illustrates the virtual environment with the torch toggled off. The user's interaction relative to these discriminators (e.g. when the torch is turned on and off, how the torch meter is managed, etc.) may be monitored for facilitating screening for and/or monitoring the condition.

In some implementations, other discriminators, in addition to the environment-based discriminators, may be evaluated. Such discriminators may be obtained from data points captured during the user's interaction with the virtual environment.

One such discriminator may facilitate evaluation of “sustained attention”, which is one of the criteria indices for ADHD inattention subtype. This discriminator may include evaluating how long a user can be engaged before making mistakes. This may be achieved by progressively increasing the speed at which the virtual character travels whilst strategically presenting obstacles to force user engagement. The forced user engagement may for example be a requirement to move the virtual character side-to-side or to jump to avoid hitting obstacles. Such a discriminator may accordingly include a number of stimulus output elements in response to which the user is required to input a satisfactory interaction instruction (i.e. an instruction to dodge the stimulus output element in the form of an obstacle). The user's ability or lack thereof to input the satisfactory instruction may facilitate screening for the condition (in this case ADHD). In some cases, failure to input the satisfactory instruction (e.g. resulting in the virtual character colliding with an obstacle) may result in resetting the segment and as a consequence resetting the speed at which the virtual character travels.

Another discriminator may facilitate evaluation of the user's ability to adhere to so-called “daily tasks” (one of the criteria indices for ADHD inattention subtype). In one exemplary implementation this may include providing the virtual character with a torch to have visibility in dark portions of the virtual environment. The torch may only work when enough tokens are collected and therefore the user is required to ensure that enough tokens are collected in order to have enough light to see them through dark portions of the virtual environment. Tokens collected (and which enable provision of light) may deplete over time with use. Such a discriminator may include a requirement to repeatedly over time attend to the performance of certain, predefined tasks and may be coupled to a functional effect or benefit and/or a cost.

Yet another discriminator may facilitate evaluation of “cognitive reasoning”. In an exemplary implementation this may for example include placing a predefined token behind an obstacle and requiring the user, through interaction instructions input into the communication device, to outlast a time delay by negotiating, hitting or otherwise overcoming the obstacle in order to obtain the token. The token may be linked to a reward to incentivize its capture (e.g. the torch fuel meter may be filled up, taking care of fuel provisions for dark portions that lie ahead). There may also be time incentives, e.g. by potentially saving time by enabling the user to focus solely on avoiding obstacles.

Yet another discriminator may facilitate evaluation of “distractibility” (one of the criteria indices for ADHD inattention subtype). This may include for example include introducing into the virtual environment one or both of auditory and visual distractor output elements (or simply ‘distractions’). Such distractions may be introduced individually or combined at specifically “random” times (from the perspective of the user, at least) throughout interaction with the virtual environment in order to test the distractibility of the user. The distractions may for example be introduced strategically at times which are likely to cause the user's navigation of the virtual character to fall fowl (e.g. resulting in the virtual character colliding with an obstacle). Such a discriminator may accordingly include a number of distractions which are configured to distract the user's attention away from stimulus output elements that are being introduced as a part of another discriminator thereby to make it more difficult for the user to input a satisfactory interaction instruction. In some cases, there may be an interrelationship between different discriminators (e.g. between stimulus output elements and distractions).

Other discriminators may for example include pausing or stopping of the virtual environment (e.g. through input of a pause or exit input element into the communication device). Such discriminators may accordingly be configured to facilitate evaluation of “avoidance of instructed task” (one of the criteria indices for ADHD inattention subtype).

As mentioned, in some implementations, the virtual environment may be purpose built for screening and monitoring one or more conditions. An example of such a virtual environment is described in the following, with reference to the schematic diagram of FIG. 2A and the screenshots of FIGS. 2B and 2D.

The virtual environment may include a number of segments, each of which may present a combination of challenges. Segments may be modelled on the DSM-V classification criteria for the particular condition (e.g. ADHD-I). Individual segments may be considered inter-linked, mini-games, which are designed to have a duration of approximately one minute each. Each segment may be followed by a subsequent segment after a brief three-second black loading screen.

In one example implementation, seven segments (numbered 0 to 6) may be provided. Segments zero and six may for example be identical and may serve as references for comparison. The segments may contain gems to collect together with a number of obstacles to avoid. The segments may be void of any other discriminators or distractors. The segments may further require a user to perform an alternative form of input so as to successfully complete the segment by hitting as few objects as possible.

Segments two and four may include auditory distractors, whereas segments three and four may include visual distractors. Segments two and four may for example facilitate evaluation of the ability of a user to realise the objectives of the segment so as to complete the segment successfully. The auditory distractions may facilitate determining the influence of such actions on the abilities of the user. In segments three and four, the user should still be able to successfully complete the segment by collecting tokens and avoiding obstacles whilst using alternative inputs. Segment four may be used to evaluate the effect of both the audio and visual distractions. The objective of this segment may be the same as that of segments two and three. Segment one may for example be designed as an empty mine tunnel, and segment five may include only a few game assets toward the end of the segment to induce a level of boredom and fatigue. Game assets are the components that fill the segment (e.g. rocks, gems, obstacles, lights sources, etc.).

Table 1 below exemplifies various assets that may be included in each of a seven game segments. It should be appreciated that table 1 below is but one example of a virtual environment and other implementations may have more or less than seven segments and other configurations of segments may be provided.

| | Pink | | Auditory | Visual | Kamikaze | |
|---|---|---|---|---|---|---|
| Segment | Gems | Obstacles | Distractors | Distractors | Gem | Tiles |
| 0 | 87 | 53 | 0 | 0 | 0 | 89 |
| 1 | 0 | 0 | 0 | 0 | 0 | 89 |
| 2 | 73 | 57 | 6 | 0 | 0 | 89 |
| 3 | 80 | 52 | 0 | 8 | 0 | 89 |
| 4 | 61 | 56 | 3 | 4 | 0 | 89 |
| 5 | 0 | 12 | 0 | 0 | 1 | 89 |
| 6 | 87 | 53 | 0 | 0 | 0 | 89 |

Segment assets may be placed at random throughout each segment with the purpose of encouraging joystick engagement for effective navigation through the mine. The random placement of assets may also strengthen the ability of the machine learning component to generalise well between segments. It is also important to note that the smallest difference in asset placement influences all the other game features.

Each segment may be played in the same setting, and may involve a virtual character in the form of a panda bear avatar travelling through a dark mine tunnel on a cart. The goal in each segment is to reach the end of the tunnel as fast as possible. The dark setting was chosen for the purpose of control by being able to limit the visual stimuli presented to the user. The controlled line of sight and the irregular presentation of response-stimuli were designed to limit anticipatory responses.

Additionally, the goal was to force a user's sustained attention for good performance. Anticipatory responses are a common feature and challenge found in the literature. This may provide a user response-based mechanism that determines the rate of presented stimuli.

From the start of each segment, the stimuli presentation rate increases incrementally, from base speed to maximum speed, as the virtual character progresses through the segment. Should the virtual character collide with an obstacle, a time penalty may be incurred: the speed of the virtual character may be reset to the base speed and the speed incrementor may be reset. Stimuli presented aim to recreate a mine tunnel setting, and may include game assets such as boundary walls, ramps, obstacles, collectables, as well as auditory and visual distractors.

Users may be required to make use of an on-screen joystick to control the side-to-side movement of the virtual character. A joystick may be preferred so as to isolate and capture user movement with the communication device's three-axis accelerometer during gameplay. Contrary to traditional methods of capturing accelerometer data, each accelerometer sample may be dependent on the position of the virtual character (as opposed to, e.g. being time dependent). For example, data points in the form of the tile number and coordinates of the virtual character and/or other game assets within the tile may be recorded. This may enable direct event-based comparison.

In one example implementation, accelerometer data may be captured thousands of times (e.g. 2262 times) for the total tiles traversed in each segment (which may number less than, e.g. 100). This may translate to a large number (e.g. 20 to 30) vector data samples per game tile. In an example implementation, the fastest segment completion time, which includes starting from base speed and without obstacle collisions, may be selected to be about 61 seconds which translates to a sampling frequency of 37.08 Hz.

To achieve the game goal, it is important to avoid stimuli presented in the form of obstacles and to respond to gem stimuli by collecting them. Collection of pink gems may increase fuel in the torch fuel meter on a unit basis. The collection of a kamikaze gem may fill the torch fuel meter completely but may require an intentional sacrifice of at least two obstacle collisions in return. The kamikaze gem may be provided to challenge the user's cognitive reasoning and decision making.

If the torch is toggled on with the torch button, the user's line of sight in the tunnel is increased. The torch fuel meter then decreases at a constant rate as long as the torch is on, which simulates real-world consequences. The torch can be toggled off with the torch button in order to conserve fuel. The on-toggle of the torch increases the range of visibility in the tunnel and simultaneously decreases the pressure on response time by making it easier to avoid obstacles and collect gems. It follows therefore that the converse is also true. Certain obstacles that the user encounters can be avoided by making use of the jump button. Both the jump and torch buttons must be utilised by the user to improve obstacle avoidance and gem collection during gameplay. This is an example of simple attention. The overall avoidance of obstacles and collection of gems requires the application of sustained attention.

Combined, these result in greater overall performance to best achieve the game goal. The video or computer game may therefore be configured to force responses from users according to their performance by automatically and continuously moving the virtual character through the mine at speeds that are influenced by game elements. Go/No-Go task stimuli may be presented in the form of gems (to be collected) and obstacles (to be avoided). The response time feature and impulsivity may be measured by the number of gems collected and missed, as well as the number of obstacle collisions and misses. The response time variability feature may be measured by the segment duration as any obstacle collisions result in a time penalty. Measurement of response time and response time variation may therefore employ a reinforced learning mechanism by rewarding the user with torch fuel when gems are collected. The user may be penalised for obstacle collisions by one or more of: an auditory injury sound from the virtual character, resetting the virtual character's speed to zero, increasing the overall segment duration and further decreasing the torch fuel meter due to the virtual character speed reset. The pause button presents the option to exit the game or return to the task.

Practically, prior to testing, users may be instructed to keep their hands and forearms from resting on or against any surface, and to keep the communication device suspended during gameplay. A tutorial may then be provided in which the user is reminded of instructions should they err. The tutorial may be configured to explain all the gameplay controls and may include in-tutorial visual cues. The tutorial level may be made up of two segments, both of which may have the same duration as the game segments. Following the tutorial phase, users may be required to play the entire game from start to finish. Users may be left to complete, in this exemplary scenario, all seven game segments without external input. The fastest possible game completion time may for example be just over seven minutes (61 seconds per segment), but it may be that poorer performing users can take considerably longer. Upon completion of the game users may receive a score for the number of gems collected during gameplay of all seven segments.

It should be appreciated that the virtual environment described above with reference to FIGS. 2A to 2D is but one exemplary virtual environment and various modifications, alterations, additions, etc. may be made thereto.

The mobile software application may further be configured to record user interaction relative to the discriminators and transmit data relating to this interaction to the server (102) via the communication network (106) for processing thereat.

The server computer (102) may be any appropriate computing device configured to perform a server role. Exemplary devices include distributed or cloud-based server computers, server computer clusters, powerful computing devices or the like. The server (102) may be configured to communicate with the communication devices (104) by way of the communication network (106) and may have access to a database (108) in which a plurality of user records as well as other data and/or information may be stored.

The database (108) may for example store data points and/or feature sets associated with a particular user in association with a user record. In some implementations, data points and/or feature sets may be categorised in the database according to a segment identifier so as to enable identification of data points and/or feature sets associated with a particular segment of the virtual environment. In some cases, feature sets of a particular segment may also be associated with a start time and an end time corresponding respectively to the time at which the relevant user began the segment and the time at which the user finished the segment.

The server computer (102) may be configured to receive data relating to user interaction relative to discriminators and to input the data into a machine learning component. The machine learning component may be configured to discriminate between users with and without the condition by identifying patterns in the data which are indicative of the presence or absence of the condition and labelling the data accordingly.

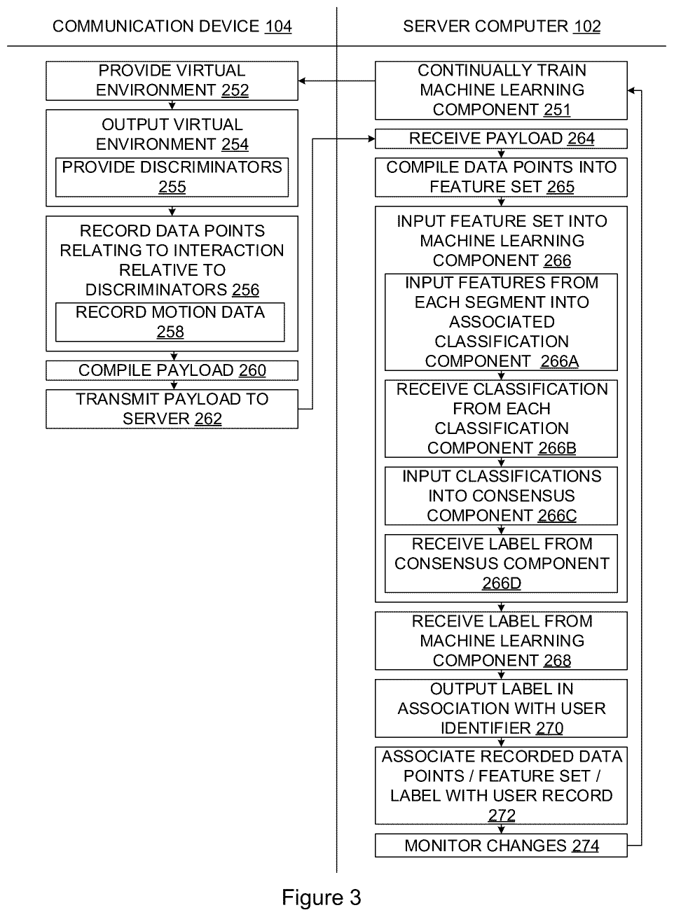

The system described above may implement a method for screening for and/or monitoring a condition. An exemplary method for screening for and monitoring a condition is illustrated in the swim-lane flow diagram of FIG. 3 in which respective swim-lanes delineate steps, operations or procedures performed by respective entities or devices.

It should be appreciated that the method may find application in one or both of screening for a condition and monitoring the condition. In some cases, monitoring the condition may include monitoring the efficacy of a drug being taken (or other therapeutic course of action) to treat the condition.

The server computer (102) may initially and in some cases continually train (251) a machine learning component with training data including pre-labelled feature sets. The pre-labelled feature sets may be feature sets which are labelled with the condition of the user who caused generation of data points from which the feature set is compiled. The pre-labelled feature sets may be associated with one or more segments of the virtual environment (i.e. it may be labelled with one or more identifiers of the segments from which it was obtained). In some cases, the pre-labelled feature sets may be associated with particular discriminators included within the virtual environment.

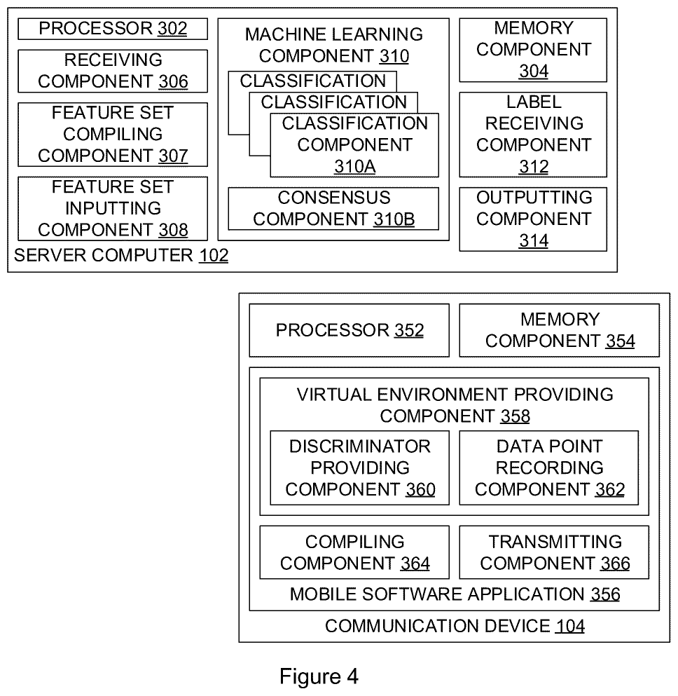

In some cases, data recorded from the plurality of communication devices (104) may be retained in the database (108) and employed for the purpose of developing an artificial intelligence type of data compilation. The machine learning component may be configured to discriminate between users with and without the condition by identifying patterns in the feature sets which are indicative of the presence or absence of the condition and labelling the feature sets accordingly. The machine learning component may implement a machine learning algorithm and may implement one or more of supervised, unsupervised and reinforcement learning. The machine learning component and training thereof is described in greater detail below, with reference to FIG. 4.

The method may include causing a user to engage in a virtual environment, which may resemble or be in the form of a computer-based game. This may be in the classroom, at home, at a medical facility or the like.

The communication device (104) may provide (252) a virtual environment (202), e.g. as illustrated in FIGS. 2A to 2B. This may include outputting (254) the virtual environment to a user of the communication device via one or more output components of the communication device (104). The virtual environment may for example be output to the user via output elements including a display and speaker of the communication device. The user may be required to interact with the virtual environment by way of a series of instructions input into the communication device (104).

As described in the foregoing, the virtual environment may include a virtual character (204) and one or more segments and the user interaction may include controlling navigation of the virtual character through and/or between segments. In some implementations, for example, the virtual environment provides an open world environment in which the user can navigate between different segments. In the open world environment, the user may be able to enter or otherwise select different segments. In some implementations, adaptive segments may be provided in which segment layout may be adapted during gameplay according to the user's measured ability for specific features. For example, segment difficulty (or degree of difficulty) or demand may be increased or decreased accordingly to maintain a specific measured feature outcome.

A number of discriminators may be provided (255) in the virtual environment. The discriminators may be environment-based discriminators, such as those described in the foregoing. In some implementations this may include providing each segment with a number of predetermined discriminators. The discriminators may be configured to facilitate discrimination between users with and without the condition.

Different types of discriminators may be provided and in some implementations different discriminators may be provided for facilitating discrimination between users with and without different conditions (i.e. different discriminators may be useful in identifying different conditions).

In some implementations, data captured from each segment may be partitioned. This may allow for specific groups of features to be tested in specific segments of the virtual environment, and for those segments to be compared and analysed independently in light of the overall data captured from the virtual environment.

For example, each segment may have the same number of tiles, resulting in a length of ±1 minute in duration when the virtual character completes a run with no obstructions. Each segment collects the data features as described herein. Individual machine learning models may be trained on the data collected from the following seven segments (one model for each segment). These models serve as individual classifiers and form part of a cross-segment validated output which is created by averaging the diagnostic feedback output of each of the Segment classifiers. This results in an averaged, cross-validated classifier. It also highlights the strength and weakness of each individual segment to produce strong discriminatory features. Furthermore, it allows for improvements to be made to specific Segments to strengthen the cross-validated classifier.

Each segment may therefore be configured to provide a criteria for identification of a condition. For example, a first segment may be configured to determine changes in concentration (ADD) and a second segment configured to determine a change in the level of activity (ADHD).

The following are examples of discriminators configured to facilitate discrimination between users with and without ADHD which may be provided in an exemplary implementation of a segment.

Some implementations may provide an open world environment having different segments in which each segment includes different discriminators which are configured to facilitate discrimination between users with and without a particular condition. Different segments may facilitate discrimination between different conditions. Discriminators may accordingly include one or more of: sustained attention; daily task; cognitive reasoning; distractibility; task avoidance; and task completion discriminators.

Discriminators may be selected based on academic literature, existing methods and the specific condition diagnostic criteria. Discriminatory values may lie within the evaluation of a combination of features and not in a single feature alone. Furthermore, in some implementations, joystick (or other I/O controller) logic may be reversed (e.g. moving a joystick left moves the virtual character right and vice versa) so as to combat advantage due to familiarity with joystick-based games.

Further to ‘gameplay’ segments of the virtual environment, there may be a separate tutorial segment. The main purpose of the tutorial segment may be to introduce the users to the virtual environment and familiarize them with the objectives and controls of the virtual environment. The data for this segment will not be captured but it may be used to evaluate whether the user is capable of following instructions.

The communication device (104) may record (256) data points relating to the user's interaction in relation to each of the number of environment-based discriminators. Recording interaction in relation to an environment-based discriminator may include recording user input and associating it with data relating environment-based discriminators that were being output, or possibly which have just been output, to the user. This may be achieved using time- and/or position-stamping of input parameters and game assets. This time- and/or position-stamping may occur at strategic times only, or alternatively throughout the segment.

Recording interaction in relation to an environment-based discriminator may have the effect of recording user input (e.g. joystick duration, button presses, accelerometer vector data values, etc.) which is specific to environment-based discriminators presented to the user in each segment. User input and environment-based discriminators may therefore be linked. This may create a relationship between environment-based discriminators and user input. This may improve performance (accuracy, specificity, selectivity, etc.) of the machine learning component and may enable multiple conditions to be discriminated with greater accuracy using a single virtual environment setup.

Recording (256) data points may thus include monitoring the user's interaction with the virtual environment and recording the effect of the user's interaction in relation to a discriminator. Recording data points may include recording and time stamping each input instruction received from the user. Recording data points may further include recording and time stamping the output of game assets including environment-based discriminators.

For example, in the case of a discriminator in the form of a stimulus output element, recording (256) parameters relating to the user's interaction relative to the stimulus output element may include recording a time stamp corresponding to the time at which the stimulus output element is presented to the user via one or more output components of the communication device. Recording (256) parameters may include recording a time stamp corresponding to the time at which the user inputs an input instruction in response to presentation of the stimulus. Recording (256) parameters may include evaluating an input instruction received in response to output of the stimulus output element against the predetermined expected instruction input. For example, in the case of the collectable item mentioned above, the predetermined expected instruction may include a series of input instructions which cause the character to move towards and ‘collect’ the collectable item. Evaluating the input instruction against this predetermined expected instruction may include evaluating whether the input instructions received into the communication device are sufficient to cause the character to move towards and collect the collectable item. In some cases, recording (256) parameters may include tracking a trajectory of the virtual character through the virtual environment in relation to the location of the stimulus output element in the virtual environment, wherein the trajectory of the virtual character is controlled by user input.

In an exemplary implementation, one or more of the following data points relating to the user's interaction may be recorded:

| | |
|---|---|
| “Age”:“12.0”, “AudioDistractions”:[“2017/12/4T13:41:6.42”, | ...], “Diagnosed”:“1”, |
| “EndDateTime”: “2017/12/4T13:41:22.11”, | “Gender”:“Female”, |

“Id”:“011358FD300C0D040029012A341DA1A0”, “JoystickCount”:“6890”, “JumpCount”:3,

| | |
|---|---|
| “KamikazeCollected”:0, “MainUser”:“0”, “Medicated”:“0”, | “ObstaclesAvoided”:11, |
| “ObstaclesHit”:“1”, | |

“PauseGamePressed”:[“2017/12/4T13:41:13.846”, “2017/12/4T13:41:15.651”, “2017/12/4T13:4

| | |
|---|---|
| 1:20.508”], | “Race”:“black”, |

“ResumePressed”:[“2017/12/4T13:41:14.764”, “2017/12/4T13:41:17.436”, “2017/12/4T13:41:21
.944”], “SegmentId”:1, “SessionId”:“20171204134044”,

| | |
|---|---|
| “StartDateTime”: “2017/12/4T13:41:4.389”, “TilesTraversed”:10, | “TokenCollected”:16, |

“TokenMissed”:4, “TorchCount”:1, “Vectors”:[“X=−0.068 Y=−0.632 Z=2.095”, “X=−0.046 Y=−

| | |
|---|---|
| 0.547 Z=2.097”,“X=−0.006 Y=−0.370 Z=2.104”, | ...], |

“VisualDistractions”:[“2017/12/4T13:41:15.600”, ... ]

Features with “, . . . ” may include multiple data point for that feature.

It should of course be appreciated that these are exemplary data points and others may be recorded too, such as timestamps when obstacles are hit so as to relate specific obstacles to the entering of specific distractors.

Similarly, recording data points relating to the user's interaction relative to a discriminator in the form of a distractor output element may include logging the time at which the distractor output elements are introduced and tracking a trajectory of the virtual character through the virtual environment in relation to the location of one or more stimulus output elements so as to be able to monitor the effect of the distraction on the user's ability to control the virtual character (and in turn to monitor the user's distractibility).

Recording data points relating to the user's interaction relative to a discriminator in the form of a cognitive reasoning discriminator may include recording data points relating to the user's performance of the required task.

In some cases, the communication device (104) may record or monitor one or more of the following: the time taken to complete the segment; a number of failures whilst interacting with the virtual environment (e.g. while playing the computer-based game); the distractibility of the user to concentrate on a particular item forming part of the segment in the presence of other items that are calculated to be a distraction and the like.

The communication device (104) may compare the results with results of an earlier determination carried out in an analogous manner at an earlier time and may evaluate the differences in order to monitor the condition (including, e.g., assessing the effectiveness of a drug or other therapeutic procedure that has been administered to, or conducted on, the user in the intervening time period).

In some implementations, recording (256) data points may include recording (258) motion data produced by motion sensors associated with the communication device. The motion data may be recorded at strategic times (e.g. during cognitive reasoning discriminator, etc.). As mentioned above, in some implementations, motion data may be dependent on the position of the virtual character so as to facilitate event-based comparison. For example, by associating motion data with the position of the character within the virtual environment, the motion data may be associated with the output of a particular environment-based discriminator.

The motion data may relate to acceleration and/or rotation data produced by an accelerometer or gyroscope respectively. The recorded motion data may be position stamped and/or time stamped to indicate a point in time at which the data was recorded. The recorded motion data may include for example, for each of the tri-axis (x, y and z), and may be processed at a later stage to determine one or more of: a minimum and maximum acceleration range, a mean value, a median value, standard deviation, variance, kurtosis, skewness, interquartile, 25th percentile, 75th percentile and the root mean square. The recorded motion data may consequently be used to distinguish between normal users and users with a particular condition, for example such as ADD/ADHD and also to monitor progress.

In some implementations, a wrist-worn vital monitoring system may be used during a training phase of artificial intelligence for the system to monitor heart rate and other vital data points of the user. A sensor in a hand glove may alternatively be provided that will detect the data points of acceleration and heart rate variability that are desired. Tactile sensors could be used as part of the game play where the child is requested to pick up certain toys (duck, sheep etc.) according to a game algorithm and a small magnet that is placed in the toy will then confirm that the toy has been successfully picked up. The degree of complexity would be determined by the age of the user.

One of the options of monitoring the level of activity is to have a handheld device, where such a device, or devices, is able to measure acceleration and could also measure heart rate and heart rate variability which could be used in an algorithm to distinguish between normal users and users with a particular condition, for example such as ADD/ADHD.

The communication device (104) may compile (260) a payload including the recorded data points, a user identifier uniquely identifying the user and optionally other data and/or information. The payload may be a data structure (including, e.g., one or more data packets) which includes the data points and any other information which may be necessary for the storage and/or transmission of the data points. The user identifier may for example be a user name having been input into the communication device at the time of commencing interaction with the virtual environment and may be capable of uniquely identifying the user to the server computer (102). In some implementations, compiling the payload may include associating the recorded data points with a discriminator in relation to which they were recorded.

The payload may for example include a mapping of the recorded data points to the corresponding discriminator. The data points may for example include: a description of the discriminator; a time stamp corresponding to the time at which the discriminator occurred and/or a duration associated with the discriminator; a position stamp relating to the position at which the discriminator is introduced and/or a position stamp relating to the position of the virtual character; a description of the user input received immediately after the occurrence of the discriminator and/or during the occurrence of the discriminator; timestamps corresponding to this user input; tracking information relating to the location/position of the virtual character in the virtual environment and/or relationship data relating to the location of the virtual character in relation to other objects/obstacles in the environment; motion data and the like. For example, in response to stimulus X, user input in the form of Y was received, which caused Z to happen to the virtual character. Timestamps may include milliseconds and some features may include multiple data points.

The communication device (104) may transmit (262) the payload to server computer (102) for processing. Transmission (262) may be via the communication network (106). It should be appreciated that in some cases, the communication device (104) may be geographically separated from the server computer (102) by a considerable distance (e.g. in another country or on another continent even).

The server computer (102) may receive (264) the payload including recorded data points, a user identifier and optionally other information and/or data from the communication device (104). The payload may be received via the communication network (106). It should be appreciated, despite only one communication device being illustrated in the method of FIG. 3, the server computer (102) may receive payloads from a plurality of communication devices (104). The plurality of communication devices may be distributed across large geographical regions.

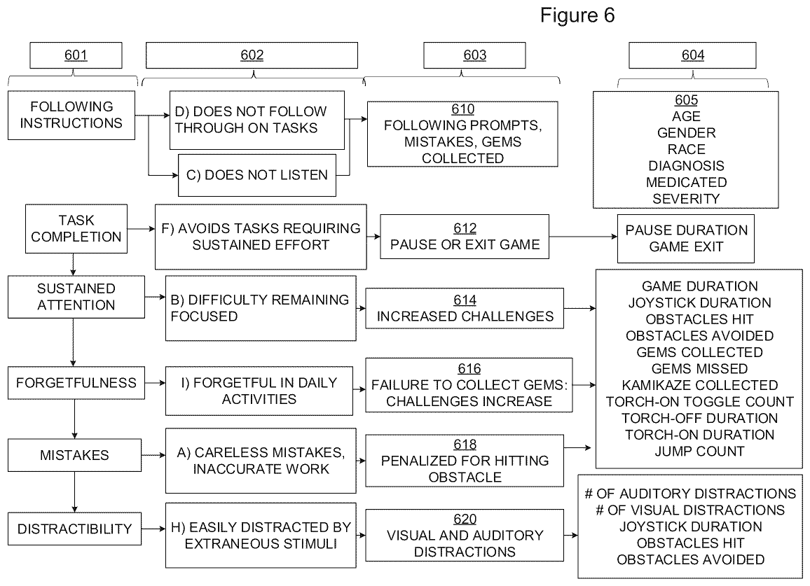

The server computer (102) may compile (265) at least a subset of the data points into a feature set. The subset of data points included in the feature set may represent first order features and compiling (265) the data points into the feature set may include processing the first order features to generate second order features and including at least a subset of the second order features together with the subset of the first order features in the feature set. FIGS. 6A and 6B, which are discussed in greater detail below, illustrate compilation of data points into feature sets and the processing of first order features to output second order features.

The server computer (102) may input (266) the feature set into a machine learning component configured to discriminate between users with and without the condition by identifying patterns in the feature set which are indicative of the presence or absence of the condition and labelling the feature set accordingly. The data points may be input in association with the user identifier.

The machine learning component may process the feature set so as to identify the presence or absence of one or more conditions based on patterns that the component is able to recognise in the data points. Doing so may include drawing on training data.

The machine learning component may include one or more classification components configured to classify the feature set based on patterns included therein. Each classification component may implement a classification algorithm, for example a neural network-, boosted decision tree- or locally deep support vector machine-based algorithm.

In some implementations, the machine learning component may include a plurality of classification components. Each of the plurality of classification components may be associated with a corresponding segment of the virtual environment and may have been trained using data points obtained from the segment of the virtual environment with which it is associated.

The feature set may be partitioned to delineate features obtained from each of the segments, and inputting the feature set into the machine learning component may include inputting (266A) features obtained from a particular segment into the associated classification component and receiving (266B) a classification from each classification component which corresponds to each of the segments. In other words features obtained during a particular segment of the virtual environment may be input into a classification component which has been trained from data obtained from that same segment.

In some implementations, the machine learning component may include a consensus component configured to evaluate the classifications of each of the classification components and to output a label based on the consensus. Inputting the feature set into the machine learning component may then include inputting (266C) each of the classifications into the consensus component and receiving (266D) a label from the consensus component which may then be output by the machine learning component. The label received from the consensus component may include a classification (e.g. normal/abnormal, ADHD-I (or other condition, as the case may be), a pointer to a disorder spectrum, etc.) and optionally a confidence measure which indicates confidence in the classification.

The server computer (102) may receive (268) a label from the machine learning component which indicates either the presence or absence of the condition. This may include receiving the label from the consensus component of the machine learning component. In some cases, the condition may be linked to a spectrum in that manifestations of the condition cover a wide range, from individuals with severe impairments to high functioning individuals who exhibit minor impairments only. An example of a condition linked to a spectrum is autism, manifestations of which range from individuals with severe impairments—e.g. who may be silent, developmentally disabled, and locked into hand flapping and rocking—to high functioning individuals who, e.g., may have active but distinctly odd social approaches, narrowly focused interests, and verbose, pedantic communication. In such a case, the label may indicate either the presence or absence of the condition by indicating a region of the spectrum with which the data points are associated.

The server computer (102) may output (270) the label in association with the user identifier. Outputting (270) the label may include associating (272) one or more of the recorded data points, the feature set and the label with a user record stored in the database (108) and linked to the user identifier. Outputting (270) the label may further include transmitting the label to the communication device (104) from which the corresponding data points were received and/or to a communication device of a medical practitioner linked to the user identifier. Transmission may be via the communication network (106).

The server computer (102) may monitor (274) changes in the recorded data points, the feature set and/or labels associated with the user record. For example, the same user may interact with the virtual environment periodically over a period of time which may result in the server computer (102) periodically receiving updated data points. The operations described above may be repeated to monitor for any changes in the data points for use in informing a medical practitioner on the efficacy of a particular drug being taken by the user or to otherwise monitor progression or regression of the relevant condition.

It should be appreciated that although FIG. 3 illustrates certain operations (in particular the inputting of a feature set into the machine learning component) being conducted at the server computer, in other implementations, some or all of these operations may be performed by the communication device. In some implementations, for example, access to the machine learning component may be provided to the communication device, which may in turn be able to compile (or obtain compilation of) data points into a feature set for input into the machine learning component.

Various components may be provided for implementing the method described above with reference to FIG. 3. FIG. 4 is a block diagram which illustrates exemplary components which may be provided by a system for screening for and monitoring a condition.

The server computer (102) may include a processor (302) for executing the functions of components described below, which may be provided by hardware or by software units executing on the server computer (102). The software units may be stored in a memory component (304) and instructions may be provided to the processor (302) to carry out the functionality of the described components. In some cases, for example in a cloud computing implementation, software units arranged to manage and/or process data on behalf of the server computer (102) may be provided remotely.

The server computer (102) may include a receiving component (306) arranged to receive a payload including recorded data points and a user identifier uniquely identifying a user from the communication device (104). The data points may relate to a user's recorded interaction relative to each of a number of discriminators provided in a virtual environment with which the user interacts and configured to facilitate discrimination between users with and without the condition.

The server computer may include a feature set compiling component (307) for compiling a feature set including at least a subset of the data points received in the payload. In some implementations the feature set compiling component may process first order features to obtain second order features and subsets of the first and second order features may be included in the feature set.

The server computer (102) may include a feature set inputting component (308) arranged to input the feature set into a machine learning component (310).

The server computer (102) may include or otherwise have access to the machine learning component (310), which may be configured to discriminate between users with and without the condition by identifying patterns in the data points and/or feature set which are indicative of the presence or absence of the condition and labelling the data points and/or feature set accordingly.

In some implementations, the machine learning component (310) may be a remotely accessible machine learning component. The machine learning component (310) may for example be a cloud-based machine learning component hosted by a third party service provider and may be accessible via an API (e.g. a web-based API).

In some implementations, the machine learning component includes one or more classification components configured to classify the feature set based on patterns included therein. The machine learning component (310) may for example include a plurality of classification components (310A). Each of the classification components may be associated with a corresponding segment of the virtual environment.

The feature set may be partitioned to delineate features obtained from each of the segments, and the machine learning component (310) may be configured to input features obtained from a particular segment into the associated classification component (310A) and to receive a classification from each classification component which corresponds to each of the segments.

Each classification component (310A) may be trained using data points obtained from the segment of the virtual environment with which it is associated. Each classification component may implement a machine learning classifier.

The machine learning classifier may be a two-class model and may be implemented using any learning approach, such as supervised learning, unsupervised learning or the like. In some cases, where a variety of different conditions are being screened for and/or monitored, a higher class model may be used. Supervised learning may be a technique used to train a two-class machine learning classifier to categorise the user data samples. This learning technique gives the machine learning classifier access to the true diagnostic condition of the users while the classifier is training how to categorise the users.

Exemplary machine learning classifiers include: averaged perception, Bayes point machine, boosted decision tree, decision forest, decision jungle, locally deep support vector machine (LDSVM), logistic regression, neural network, deep neural network and a support vector machine. LDSVM-based classifiers, in combination with a consensus component, may be more effective in cases where large volumes of training data (e.g. in the form of pre-labelled feature sets) are not readily available. Deep neural network-based classifiers may be more effective in cases where large volumes of training data are available and may be able to cluster participants with co-existing disorders.

In some implementations, a multi-classifier approach may be implemented in which each segment of the virtual environment has a corresponding machine learning classifier. The average from these individual classifiers may then constitute the final user classification. In the case of a virtual environment having seven segments for example, this may entail providing at most seven classifiers (but in some cases less, for example where, as discussed above, selected segments directed at influencing the user are excluded). In some cases, all classifier techniques may be trained and adjusted on a particular segment (e.g. segment zero) to determine the optimal performing classifier. The optimal performing classifier may then be selected to train on each of the remaining (e.g. five) virtual environment segments individually.

In other implementations, a skeletal classifier model approach may be taken. A skeletal classifier may be configured to generalise on segment data, including intersegment variation on all its features.

In such an implementation, the machine learning component (310) may further include a consensus component (310B) configured to evaluate the classifications of each of the classification components (310A) and output a label based on the consensus. The machine learning component may be configured to input each of the classifications received from the classification components into the consensus component (310B) and receive a label from the classification component.

This may include providing a consensus component (310B) which executes a consensus algorithm within the machine learning component that cumulatively evaluates virtual environment segments and provides a single consensus classification output, C_{f}, as well as a consensus confidence score, C_{c}. An exemplary consensus algorithm has the following form:

$${C}_{f} = {\sum }_{i = 0}^{n} {c}_{i} \\ {C}_{c} = \frac{\vert {C}_{f} \vert}{n} \times 100$$

where i represents the position of the segment in the virtual environment, c represents the classification of the segment (e.g., normal=1, abnormal=−1), n represents the total number of segments included in the analysis, and C_{f} represents the final consensus classification. The consensus confidence score, C_{c}, may be a percentage value indicating the degree of consensus. For user classification, the following is applicable:

$$if {C}_{f} > 0 ; classification = abnormal \\ if {C}_{f} < 0 ; classification = normal$$

In some implementations of the skeletal model approach, a number of machine learning classifiers may be trained and adjusted on a number of (e.g. 5) segments. The optimal performing classifier may then be selected for integration with the consensus algorithm to provide a final classification of users. In some implementations, filter-based feature selection may be used to determine the most significant features according to Pearson's Correlation and a stepwise feature removal may be implemented according to the Pearson's correlation to improve model performance.

The server computer (102) may include a label receiving component (312) arranged to receive a label from the machine learning component (310) indicating either the presence or absence of the condition.

The server computer (102) may include a label outputting component (314) arranged to output the label in association with the user identifier.

The communication device (104) may include a processor (352) for executing the functions of components described below, which may be provided by hardware or by software units executing on the communication device (104). The software units may be stored in a memory component (354) and instructions may be provided to the processor (352) to carry out the functionality of the described components. In some cases, for example in a cloud computing implementation, software units arranged to manage and/or process data on behalf of the communication device (104) may be provided remotely.

Some or all of the components may be provided by a mobile software application (356) downloadable onto and executable on the communication device (104). The mobile software application (356) may resemble or be in the form of a video game or computer game. The mobile software application (356) may provide a Paediatrics Attention Deficit Disorder App (PANDA) and may operate on different levels of sophistication. Mathematical algorithms may be configured to track the progression through segments and decipher the specific criteria according to international cognitive and behavioural guidelines.

The mobile software application (356) may include a virtual environment providing component (358) arranged to provide a virtual environment which is output to a user via one or more output components of the communication device and with which the user is required to interact by way of a series of instructions input into the communication device.

The mobile software application (356) may include a discriminator providing component (360) arranged to include a number of discriminators in the virtual environment. The discriminators may be environment-based discriminators and may facilitate discrimination between users with and without the condition based on the user's interaction with the virtual environment relative to the discriminator.

The mobile software application (356) may include a data point recording component (362) arranged to record parameters relating to the user's interaction in relation to each of the number of discriminators.

The mobile software application (356) may include a compiling component (364) arranged to compile a payload including the recorded data points and a user identifier which may uniquely identify the user (and optionally additional data/information).

The mobile software application (356) may include an outputting component (366) arranged to output the payload for input into a machine learning component configured to discriminate between users with and without the condition by identifying patterns in the data points. The outputting component may include a transmitting component for transmitting the payload to the server computer for processing thereat.

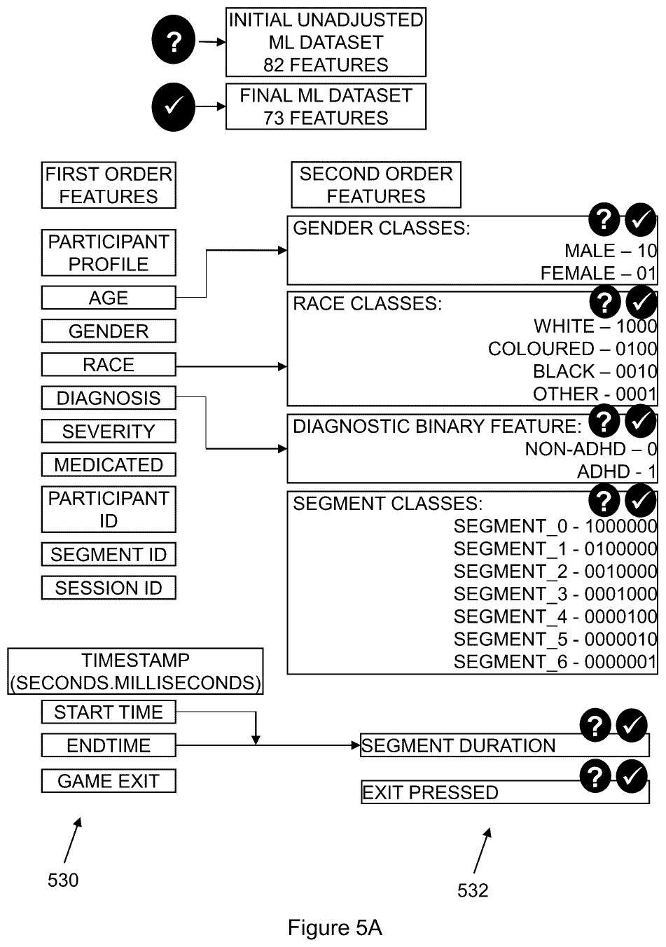

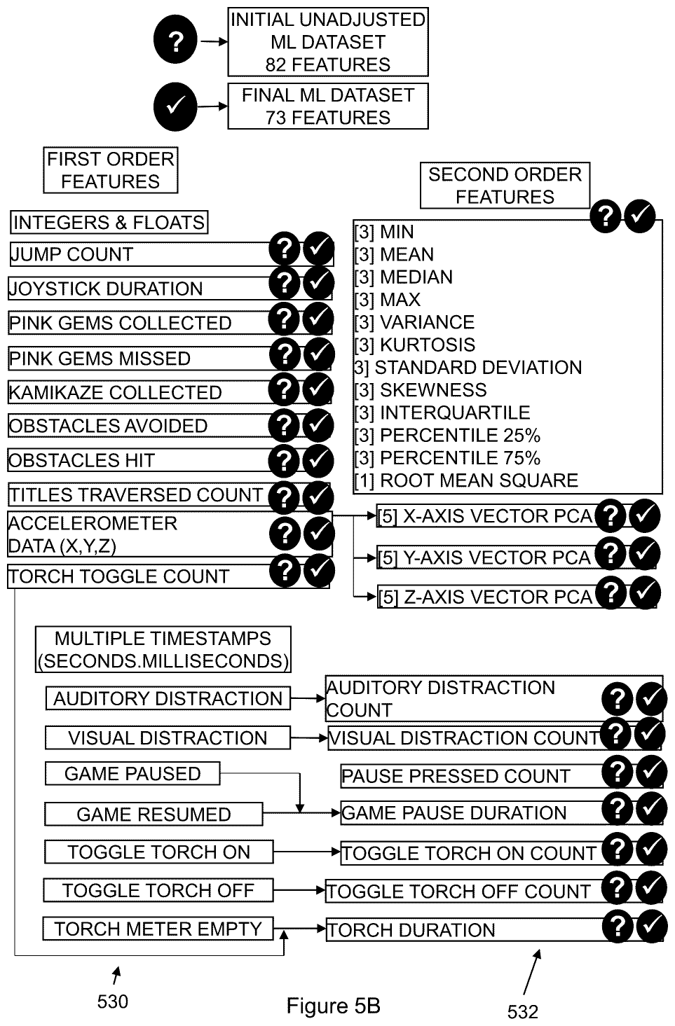

FIGS. 5A and 5B illustrate compilation of data points into a feature set, including the processing of first order features to produce second order features. First order features (530) may be extracted or recorded by the communication device during gameplay. Second-order features (532) may be generated post-gameplay.

Second order features may be created or derived from first-order features through mathematical computations and keeping track of more in-depth virtual environment logic. Second-order features may be created by transforming first-order features from integer values to classes, to count event occurrences captured by first-order features, to perform computations on first-order feature values or the like.

In some cases, user profile features such as gender, race and unique identifier may be transformed into classes to prevent the addition of a weighting for any specific value and to instead indicate a class difference. A diagnosis feature may be transformed into a binary class feature. Multiple first-order features may be captured by means of timestamps as events occurred during interaction with the virtual environment (or ‘gameplay’). These timestamps may be converted into a single binary feature (e.g. Game Exit to Exit Pressed) or used to calculate durations (e.g. Start Time and End Time to determine Segment Duration). Certain timestamps may be used to determine the number of times a feature occurred (e.g. Auditory Distractions to attain Auditory Distraction Count).

With reference to the exemplary virtual environment of FIG. 2, multiple first-order features, of both integer and timestamp format, may for example be required to calculate the Torch Duration second-order feature. These first-order timestamp features may include Torch Toggle On, Torch Toggle Off and Torch Meter Empty. The requisite integer feature may be Torch Toggle Count. The torch is a feature of the game that is unaffected by transitions between segments. Due to this continuous mechanism, multiple torch state conditions had to be checked for the array of timestamps.

Compilation of data points into a feature set may include performing PCA. In some implementations, PCA may be performed on the three-axis accelerometer data to reduce the dimensionality of the vector data features. The PCA may be performed on each axis individually (axes x, y and z) so as to improve classification performance. Missing vector data values may be replaced with a zero value as the accelerometer vector values may range between negative and positive real numbers. A standard scaler may be used before PCA, which may normalise the vector data (standard deviation=1, mean=0).

In some implementations, a statistical feature set may be created from the accelerometer data captured during gameplay. FIG. 5B illustrates an example 34 second-order statistical features created from the three-axis accelerometer data. The Root Mean Square feature may be calculated using all three axes.

Question marks indicate initially proposed features and ticks indicate features used to train the machine learning classifier(s).

In some implementations, a newly generated feature set including original gameplay data points and the generated features may be stored in a different table on the same database. The newly generated feature set may then be used to train the machine learning component. In some implementations, a single machine learning component may be trained for each individual segment. Furthermore, a cross-segment machine learning component may be implemented and trained using the data captured across all of the aforementioned segments. The strength of the patterns identified in the data, which may be indicative of the presence or absence of the condition, of the cross-segment machine learning component may be compared to the strength of the patterns identified for each individual segment by the machine learning component.

In some implementations, an API may be generated for each machine learning component as well as the cross-segment machine learning component. The API generated for the machine learning component may provide feedback on the patterns identified in each individual segment of the application. The feedback may be averaged such that a single cross-segment validated output may be generated. The API of the cross-segment machine leaning component may provide feedback regarding the identified data points to the user of the device and serves as a cross-game classifier, able to classify users tested on new/different games, provided that the new games extract the same features.

Once the initial training of the machine learning component and the generation of the APIs have been completed, the APIs may be incorporated into the game and updated as the user data increases. New machine learning components may be routinely trained and updated APIs may be generated. The updated APIs may be used for more accurate discrimination between users.

A user facing front-end may be used to provide a specific user, such as a medical practitioner, access to specific raw data of another user, such as a patient, and specific API revisions based on a user profile.

FIG. 6 illustrates an exemplary mapping of features to DSM-V criteria according to aspects of the present disclosure. The mapping may be in the form of a matrix which may include a summary (601) column which summarizes the DSM-V criteria into symptoms presented by patients with ADHD inattention subtype.

The DSM-V Diagnostic Criteria column (602) gives the criteria points included for quantification in the described systems and methods.

The GOAL column (603) describes the overall goal that the patient is to achieve in the game, as well as how the described system and method attempt to extract specific data features. The goal is to complete the game in the shortest time possible, whilst missing as many obstacles as possible and collecting as many collectable items (or ‘gems’) as possible. A timer element is used to assess whether the goal is met and can, for example, be a time-based milestone which is achievable by the user through dedication of sufficient time and configured to time continued interaction with the virtual environment. To test the user's ability to follow instructions (610), the user must follow all the prompts during the tutorial as the tutorial visually explains the game interface. This is also reflected in the number of mistakes made and collectable items collected during the game. To assess whether the user can complete tasks (612), the user must complete the game without using the pause/exit elements which can, for example, be Game Pause or Exit buttons. Vision is intentionally limited to force sustained attention and the goal (614) is to avoid obstacles and collect as many collectable items as possible whilst the virtual character's speed increases over time. Also, the virtual character automatically reverts back to the centre lane, requiring sustained engagement with the joystick to avoid obstacles. To assess the user's level of forgetfulness (616), the user must aim to collect as many collectable items as possible as failure to do so will result in the torch fuel meter running empty. As a result, distance of sight will be severely limited, and ultimately obstacle hits will occur. Hitting obstacles are regarded as mistakes made by the user (618) and will reset the virtual character's speed, thereby increasing the duration of gameplay. Finally, the user's ability to avoid distractions is assessed by introducing visual and auditory distractions (620) in certain segments with the intention of straining sustained attention and forcing mistakes.

The data features column (604) and extra data features box (605) illustrate the data recorded by the communication devices.

Aspects of this disclosure may accordingly provide a video game or computer game which is generally designed so that it highlights the variables to be determined or the attributes to be monitored. Custom designed computer-based games are therefore envisaged. The computer-based games may be aimed at different age groups, for example three age groups of 4-6 years; 7-12 years; and 13-17 years. The game may further be void of any written language so as to have global utility. The complexity of the game may increase with each age group and could include arithmetic and mathematical challenges. The computer-based games may have one of more of the following three outcomes; the time taken to complete the game as well as inter-game segments should be recorded; the number of failures to perform a certain task should be recorded; for instance touching a green coloured spaceship before it disappears from the screen; and, the ability of the user to concentrate on a specified item whilst lights flash or other objects are illuminated to attract attention away from the specified item.

The results of a computer-based game may be produced in the form of a quantitative measurement for each of the outcomes. The outcomes may be used in a number of algorithms. Each algorithm may be tested against the normal and patient populations to ascertain which algorithm is the most sensitive in distinguishing between them. Applying artificial intelligence (AI) a combination of algorithms could be applied to optimize the efficacy of the computer-based game.

The ADHD screening tool has been designed and developed to include a feature set and a machine learning algorithm that serves as a skeleton for any game layout or visual overlay within certain limits. This was implemented to enable the possibility for dynamically changing game segments whilst still providing classification accuracy. Therefore, each game segment can be an interchangeable mini-game used in the overall classification of a participant. Random placement of game assets was implemented to establish a framework according to which future games can be developed. In principle, the seven segments constitute seven mini-games with the same feature set but different values for each of the features (e.g. the number of obstacles, gems and distractions). By retaining elements such as the number of segment tiles and game logic, any segment can be replaced by a different visual overlay (e.g. a car on a racetrack at night). If the segment measures the same features, that segment could potentially be used to classify participants according to the same underlying skeletal MLC that was trained on the five segments designed and developed in this study. Due to the classifier's ability to generalise well across the five gameplay segments included during the training of the classifier, there is scope to affirm the skeletal feature and game structure suggested. The classifier generalises well across (e.g., all) gameplay segments.

Aspects of this disclosure provide software in the form of a game app, which can be downloaded onto a portable communication device. The game app may be developed for use by children and adolescents, aged between, for example, 6 and 12 and may implement sound methods of evaluating neuropsychiatric disorders. Implementation of artificial intelligence may serve as a mechanism for analysing clinical data. Aspects of the disclosure may employ cross-segment diagnostic validation and/or cross-game diagnostic validation. In some cases, aspects of the disclosure may form a part of one of many diagnostic games in a larger open world game and in-game environment and tasks may be designed amongst others to challenge poor attention and force sustained attention.

The method described herein may be used by paediatricians and psychologists to aid in the identification of ADD/ADHD (and other neuro-developmental disorders) as well as to distinguish between the two sub groups (and other disorders). Population studies may be carried out in order to ascertain the incidence of ADD/ADHD and other conditions. Drug research may be carried out to determine the effectiveness of a new or current drug. Medical insurance may be made responsible for the costs of drugs within the specified population of ADD/ADHD patients.

Evaluation of drug effectiveness may be employed by various persons for different purposes. Psychiatrists or psychologists may use the method to determine effectiveness in a treatment. School teachers could use the method to ascertain drug effectiveness. Parents may be able to use the method to ascertain drug effectiveness and to monitor drug compliance. Aspects of this disclosure may find application in early screening (e.g. for use by parents, teachers and carers; monitoring the effect of medication; serving as an additional screening tool for use by medical professionals (paediatricians, clinical psychologists, etc.) and the like.

The system and method described herein may increase access to what would otherwise have been specialist techniques and may find particular application in rural and/or developing regions. Further, using the described system and method, monitoring and identification of conditions may be conducted in a setting in which the child feels natural and comfortable (as opposed to, e.g., in front of a specialist medical device, in a clinic, etc.).

In experimentation using a system and method described herein, a test group comprised 39 participants including 31 ADHD inattentive subtype (ADHDI) (25 male, 6 female) and eight non-ADHD control group (6 male, 2 female) between the ages of six and twelve years, with a mean age of 9.02 years (standard deviation=1.88). A 190-fold cross-validation model was used for each classifier. The 190-fold samples include data from segments 0, 2, 3, 4, 6 for 38 of the 39 participants. Data from segments 1 and 5 were removed during the training of the classifiers as these segments served to strain the sustained attention of the participants. Additionally, given the layout of these (e.g., two) segments 1 and 5, (e.g., very) little quantitative discriminatory value was found. This was confirmed by the increase in the classification performance of the classifiers after the removal of these two segments. Data from the five gameplay segments of the 39th participant were kept as hold-out samples for manual verification. To classify a participant as either neurotypical or ADHDI, of the nine evaluated machine learning classifiers (MLCs), the three MLCs with the highest area-under-the-curve (AUC) and sensitivity-to-specificity ratio were selected for further evaluation. The sensitivity indicates the number of correctly identified ADHDI participants with respect to the total number of ADHDI participants. The specificity indicates the number of correctly identified neurotypical participants with respect to the total number of neurotypical participants. These two parameters can be used to determine a (e.g., crude) estimate of the AUC by taking the average of their combined values, as calculated from the confusion matrix, at a given threshold. However, a more accurate result is obtained by integrating over the receiver operating characteristic (ROC) curve. The results in Table 2 indicate the performance of the consensus algorithm implemented on the top three performing MLCs. The corresponding 95% confidence intervals (CI) for these MLCs are presented in Table 3.

TABLE 2
Performance metrics for the top three adjusted MLCs (190 participant samples).

| Two-Class Classifier | Accuracy % | Sensitivity % | Specificity % | AUC |
|---|---|---|---|---|
| Boosted Decision Tree | 80.5 | 96.7 | 20 | 0.806 |
| Neural Network | 78.4 | 82.7 | 62.5 | 0.836 |
| LDSVM | 91.1 | 92.9 | 82.9 | 0.942 |

TABLE 3
95% Confidence intervals (CI) for the top three MLCs.

| Two-class classifier | 95% CI (Sensitivity) | 95% CI (Specificity) |
|---|---|---|
| Boosted Decision Tree | [93.8%, 99.5%] | [7.6%, 32.4%] |
| Neural Network | [76.4%, 89%] | [49.2%, 75.8%] |
| LDSVM | [88.9%, 96.9%] | [70.4%, 95.3%] |

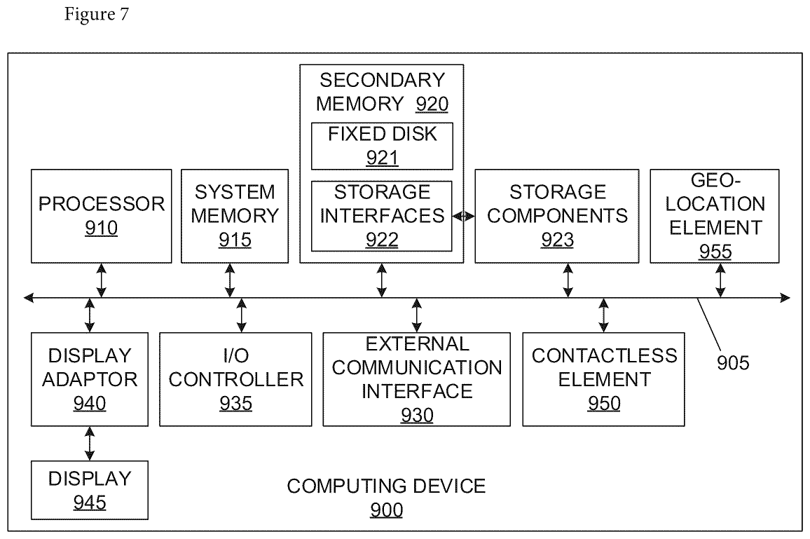

FIG. 7 illustrates an example of a computing device (900) in which various aspects of the disclosure may be implemented. The computing device (900) may be embodied as any form of data processing device including a personal computing device (e.g. laptop or desktop computer), a server computer (which may be self-contained, physically distributed over a number of locations), a client computer, or a communication device, such as a mobile phone (e.g. cellular telephone), satellite phone, tablet computer, personal digital assistant or the like. Different embodiments of the computing device may dictate the inclusion or exclusion of various components or subsystems described below.

The computing device (900) may be configured for storing and executing computer program code. The various participants and elements in the previously described system diagrams may use any number of subsystems or components of the computing device (900) to facilitate the functions described herein. The computing device (900) may include subsystems or components interconnected via a communication infrastructure (905) (for example, a communications bus, a network, etc.). The computing device (900) may include one or more processors (910) and at least one memory component in the form of computer-readable media. The one or more processors (910) may include one or more of: CPUs, graphical processing units (GPUs), microprocessors, field programmable gate arrays (FPGAs), application specific integrated circuits (ASICs) and the like. In some configurations, a number of processors may be provided and may be arranged to carry out calculations simultaneously. In some implementations various subsystems or components of the computing device (900) may be distributed over a number of physical locations (e.g. in a distributed, cluster or cloud-based computing configuration) and appropriate software units may be arranged to manage and/or process data on behalf of remote devices.

The memory components may include system memory (915), which may include read only memory (ROM) and random access memory (RAM). A basic input/output system (BIOS) may be stored in ROM. System software may be stored in the system memory (915) including operating system software. The memory components may also include secondary memory (920). The secondary memory (920) may include a fixed disk (921), such as a hard disk drive, and, optionally, one or more storage interfaces (922) for interfacing with storage components (923), such as removable storage components (e.g. magnetic tape, optical disk, flash memory drive, external hard drive, removable memory chip, etc.), network attached storage components (e.g. NAS drives), remote storage components (e.g. cloud-based storage) or the like.

The computing device (900) may include an external communications interface (930) for operation of the computing device (900) in a networked environment enabling transfer of data between multiple computing devices (900) and/or the Internet. Data transferred via the external communications interface (930) may be in the form of signals, which may be electronic, electromagnetic, optical, radio, or other types of signal. The external communications interface (930) may enable communication of data between the computing device (900) and other computing devices including servers and external storage facilities. Web services may be accessible by and/or from the computing device (900) via the communications interface (930).

The external communications interface (930) may be configured for connection to wireless communication channels (e.g., a cellular telephone network, wireless local area network (e.g. using Wi-Fi™), satellite-phone network, Satellite Internet Network, etc.) and may include an associated wireless transfer element, such as an antenna and associated circuitry. The external communications interface (930) may include a subscriber identity module (SIM) in the form of an integrated circuit that stores an international mobile subscriber identity and the related key used to identify and authenticate a subscriber using the computing device (900). One or more subscriber identity modules may be removable from or embedded in the computing device (900).

The external communications interface (930) may further include a contactless element (950), which is typically implemented in the form of a semiconductor chip (or other data storage element) with an associated wireless transfer element, such as an antenna. The contactless element (950) may be associated with (e.g., embedded within) the computing device (900) and data or control instructions transmitted via a cellular network may be applied to the contactless element (950) by means of a contactless element interface (not shown). The contactless element interface may function to permit the exchange of data and/or control instructions between computing device circuitry (and hence the cellular network) and the contactless element (950). The contactless element (950) may be capable of transferring and receiving data using a near field communications capability (or near field communications medium) typically in accordance with a standardized protocol or data transfer mechanism (e.g., ISO 14443/NFC). Near field communications capability may include a short-range communications capability, such as radio-frequency identification (RFID), Bluetooth™, infra-red, or other data transfer capability that can be used to exchange data between the computing device (900) and an interrogation device. Thus, the computing device (900) may be capable of communicating and transferring data and/or control instructions via both a cellular network and near field communications capability.

The computer-readable media in the form of the various memory components may provide storage of computer-executable instructions, data structures, program modules, software units and other data. A computer program product may be provided by a computer-readable medium having stored computer-readable program code executable by the central processor (910). A computer program product may be provided by a non-transient computer-readable medium, or may be provided via a signal or other transient means via the communications interface (930).

Interconnection via the communication infrastructure (905) allows the one or more processors (910) to communicate with each subsystem or component and to control the execution of instructions from the memory components, as well as the exchange of information between subsystems or components. Peripherals (such as printers, scanners, cameras, or the like) and input/output (I/O) devices (such as a mouse, touchpad, keyboard, microphone, touch-sensitive display, input buttons, speakers and the like) may couple to or be integrally formed with the computing device (900) either directly or via an I/O controller (935). One or more displays (945) (which may be touch-sensitive displays) may be coupled to or integrally formed with the computing device (900) via a display (945) or video adapter (940).

The computing device (900) may include a geographical location element (955) which is arranged to determine the geographical location of the computing device (900). The geographical location element (955) may for example be implemented by way of a global positioning system (GPS), or similar, receiver module. In some implementations the geographical location element (955) may implement an indoor positioning system, using for example communication channels such as cellular telephone or Wi-Fi™ networks and/or beacons (e.g. Bluetooth™ Low Energy (BLE) beacons, iBeacons™, etc.) to determine or approximate the geographical location of the computing device (900). In some implementations, the geographical location element (955) may implement inertial navigation to track and determine the geographical location of the communication device using an initial set point and inertial measurement data.

The foregoing description has been presented for the purpose of illustration; it is not intended to be exhaustive or to limit the disclosure to the precise forms disclosed. Persons skilled in the relevant art can appreciate that many modifications and variations are possible in light of the above disclosure.

Any of the steps, operations, components or processes described herein may be performed or implemented with one or more hardware or software units, alone or in combination with other devices. In one embodiment, a software unit is implemented with a computer program product comprising a non-transient computer-readable medium containing computer program code, which can be executed by a processor for performing any or all of the steps, operations, or processes described. Software units or functions described in this application may be implemented as computer program code using any computer language such as, for example, Java™, C++, or Perl™ using, for example, conventional or object-oriented techniques. The computer program code may be stored as a series of instructions, or commands on a non-transitory computer-readable medium, such as a random access memory (RAM), a read-only memory (ROM), a magnetic medium such as a hard-drive, or an optical medium such as a CD-ROM. Any such computer-readable medium may also reside on or within a single computational apparatus, and may be present on or within different computational apparatuses within a system or network.

Flowchart illustrations and block diagrams of methods, systems, and computer program products according to embodiments are used herein. Each block of the flowchart illustrations and/or block diagrams, and combinations of blocks in the flowchart illustrations and/or block diagrams, may provide functions which may be implemented by computer readable program instructions. In some alternative implementations, the functions identified by the blocks may take place in a different order to that shown in the flowchart illustrations.

The language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of embodiments herein is intended to be illustrative, but not limiting, of the scope of the disclosure, which is set forth in the following claims.

Finally, throughout the specification and claims unless the contents requires otherwise the word ‘comprise’ or variations such as ‘comprises’ or ‘comprising’ will be understood to imply the inclusion of a stated integer or group of integers but not the exclusion of any other integer or group of integers.

1. A computer-implemented method for machine learning modelling of user interface interaction comprising:
training a collection of machine learning algorithms using training data to discriminate between users with and without a neurological condition by identifying patterns in a feature set which are indicative of the presence or absence of the condition and labelling the feature set accordingly, wherein the collection of machine learning algorithms includes a locally deep support vector machine-based algorithm and a consensus algorithm; the training including:
receiving, from a mobile communication device on which a virtual environment and a plurality of environment-based discriminator events are output via a multi-touch-sensitive display thereof, a payload including recorded data points having been generated by recording and time stamping each user interface input instruction input into the mobile communication device via the multi-touch-sensitive display by a user of the mobile communication device and by recording and time stamping the output of the environment-based discriminator events such that the recorded data points are mapped to the discriminator events and relate to the user's interaction with the mobile communication device in relation to each of the plurality of environment-based discriminator events;
compiling at least a subset of the recorded data points into a feature set, wherein the feature set is designed to define features used to classify the neurological condition, wherein the feature set is partitioned to delineate features obtained from each of one or more segments of the virtual environment, wherein each of the one or more segments includes one or more of the plurality of environment-based discriminator events designed to test an aspect of the neurological condition; and
learning from the compiled feature set for a segment of the one or more segments by reference to a provided pre-labelled feature set labelled with a condition of a user who caused generation of data points from which the pre-labelled feature set is compiled.

2. The method of claim 1, including labelling the feature set with the condition of the user who caused generation of data points from which the feature set is compiled to generate the pre-labelled feature set, wherein the pre-labelled feature set is associated with one or more segments of the virtual environment, wherein the pre-labelled feature set is labelled with one or more identifiers of the segments from which it was obtained.

3. The method of claim 1, including continually training the collection of machine learning algorithms with classification feedback.

4. The method as claimed in claim 1, wherein compiling at least a subset of the recorded data points into the feature set includes the subset of recorded data points representing first order features and wherein the method includes:
processing the first order features to generate second order features; and,
including at least a subset of the second order features together with the subset of the first order features in the feature set.

5. The method as claimed in claim 1, wherein the locally deep support vector machine-based algorithm is configured to classify the feature set based on patterns included therein, wherein the locally deep support vector machine-based algorithm processes the features obtained from each of the one or more segments to obtain the classification which corresponds to the segment of the one or more segments.

6. The method as claimed in claim 1, wherein training includes training the locally deep support vector machine-based algorithm using data points obtained from each of the one or more segments.

7. The method of claim 1, including a diagnostic method of:
receiving, from a mobile communication device on which the virtual environment and environment-based discriminator events are output via a display thereof, a payload including recorded data points and a user identifier uniquely identifying a user-under-test, the recorded data points having been generated by recording and time stamping each user interface input instruction received from the user-under-test and by recording and time stamping the output of the environment-based discriminator events such that the recorded data points are mapped to the discriminator events and relate to the user's interaction with the mobile communication device in relation to each of the plurality of environment-based discriminator events;
compiling at least a subset of the recorded data points into a feature set, wherein the feature set is designed to define features used to classify the neurological condition, wherein the feature set is partitioned to delineate features obtained from each of the one or more segments;
processing the feature set by the locally deep support vector machine-based algorithm, including:
processing features obtained from each of the one or more segments by the locally deep support vector machine-based algorithm to obtain a classification for that segment;
processing each of the classifications by the consensus algorithm to determine a label indicating either the presence or absence of the condition; and,
outputting the label in association with the user identifier, including transmitting, via a communication network, the label to one or both of the mobile communication device from which the data points are received and to a communication device of a medical practitioner linked to the user identifier.

8. The method as claimed in claim 7, wherein the method includes:
associating one or more of the recorded data points, the feature set and the label with a user record linked to the user identifier; and,
monitoring changes in the recorded data points and labels associated with the user record.

9. The method as claimed in claim 7, wherein the condition is linked to a spectrum and the label indicates either the presence or absence of the condition by indicating a region of the spectrum with which the feature set is associated.

10. The method as claimed in claim 1, including:
compiling the payload including the recorded data points and a user identifier with the recorded data points mapped to the discriminator events; and
transmitting the payload to a server computer or processing the payload on the mobile communication device for compilation into the feature set for the collection of machine learning algorithms, wherein the feature set represents measured features used by the collection of machine learning algorithms to classify users with and without the neurological condition by identifying patterns in the feature sets.

11. A computer-implemented method for machine learning modelling of user interface interaction comprising:
outputting a virtual environment on a mobile communication device to a user, the virtual environment being output to the user via one or more output components of the mobile communication device including a multi-touch-sensitive display with which the user interacts by inputting a series of instructions into at least one input interface including the multi-touch-sensitive display of the mobile communication device;
outputting a plurality of environment-based discriminator events in the virtual environment;
recording data points relating to the user's interaction with the mobile communication device in relation to each of the discriminator events including recording and time stamping each user interface input instruction input into the mobile communication device via the multi-touch-sensitive display by the user and recording and time stamping the output of the environment-based discriminator events such that the recorded data points are mapped to the discriminator events and relate to the user's interaction with the mobile communication device in relation to each of the plurality of environment-based discriminator events; and
sending a payload including the recorded data points to a collection of trained machine learning algorithms for discriminating between users with and without a neurological condition, from the mobile communication device.

12. The method as claimed in claim 11, wherein the virtual environment includes one or more segments, wherein different segments include different environment-based discriminator events for testing an aspect of the neurological condition.

13. The method as claimed in claim 12, wherein the virtual environment includes a virtual character, wherein each of the one or more segments includes a plurality of discriminator events for testing an aspect of the neurological condition.

14. The method as claimed in claim 13, wherein the user interaction includes controlling navigation of the virtual character through a segment of the one or more segments.

15. The method as claimed in claim 14, wherein one or more segments of the one or more segments are provided in the form of a mini-game and include a number of difficulty levels associated therewith.

16. The method as claimed in claim 15, wherein each of the environment-based discriminator events includes one or more of:
a stimulus output element provided in the virtual environment and output from the mobile communication device to the user, wherein the stimulus output element is configured to prompt a predetermined expected instruction input into the mobile communication device by the user;
a distractor output element provided in the virtual environment and output from the mobile communication device to the user, the distractor output element being configured to distract the user from required interaction with the virtual environment; and
a pause or exit input element configured upon activation to pause or exit the virtual environment.

17. The method as claimed in claim 16, wherein recording data points relating to the user's interaction in relation to the environment-based discriminator event are provided in the form of the stimulus output element includes one or more of:
recording a time stamp corresponding to a time at which the stimulus output element is output from the mobile communication device to the user;
recording a time stamp corresponding to a time at which the user inputs an input instruction in response to the output of the stimulus output element; and
evaluating the input instruction received in response to the output of the stimulus output element against the predetermined expected instruction input.

18. The method as claimed in claim 17, wherein the method is conducted by a mobile software application downloadable onto and executable on the mobile communication device, wherein the mobile software application is downloadable from an application repository maintained by a third party, and wherein the mobile software application provides the virtual environment in the form of a computer game.

19. The method as claimed in claim 18, wherein recording data points includes recording motion data produced by motion sensors associated with the mobile communication device for recording movement of the mobile communication device, wherein the motion data relates to acceleration and/or rotation data produced by an accelerometer or gyroscope respectively, wherein the recorded motion data is position-stamped and/or time-stamped.

20. A system for machine learning modelling of user interface interaction comprising: a non-transitory computer-readable storage medium; and one or more processors coupled to the non-transitory computer-readable storage medium, wherein the non-transitory computer-readable storage medium comprises program instructions that, when executed on the one or more processors, cause the system to perform operations comprising:
training a collection of machine learning algorithms using training data to discriminate between users with and without a neurological condition by identifying patterns in a feature set which are indicative of the presence or absence of the condition and labelling the feature set accordingly, wherein the collection of machine learning algorithms includes a locally deep support vector machine-based algorithm and a consensus algorithm; the training including:
receiving, from a mobile communication device on which a virtual environment and a plurality of environment-based discriminator events are output via a multi-touch-sensitive display thereof, a payload including recorded data points having been generated by recording and time stamping each user interface input instruction input into the mobile communication device via the multi-touch-sensitive display by a user of the mobile communication device and by recording and time stamping the output of the environment-based discriminator events such that the recorded data points are mapped to the discriminator events and relate to the user's interaction with the mobile communication device in relation to each of the plurality of environment-based discriminator events;
compiling at least a subset of the recorded data points into a feature set, wherein the feature set is designed to define features used to classify the neurological condition, wherein the feature set is partitioned to delineate features obtained from each of one or more segments of the virtual environment, wherein each of the one or more segments includes one or more of the plurality of environment-based discriminator events designed to test an aspect of the neurological condition; and
learning from the compiled feature set for a segment of the one or more segments by reference to a provided pre-labelled feature set labelled with a condition of a user who caused generation of data points from which the pre-labelled feature set is compiled.
